(19) United States
(12) Patent Application Publication
(10) Pub. No.: US 2009/0130167 A1
(43) Pub. Date: May 21, 2009

(54) Drug Delivery Device and Method for Use with Prosthetic Device Implantation

(75) Inventors: Kurt G. Shelton, Woburn, MA (US); Scott W. James, Danvers, MA (US)

Correspondence Address: SUTHERLAND ASBILL & BRENNAN LLP, 999 PEACHTREE STREET, N.E., ATLANTA, GA, 30309, US

(73) Assignee: MICROCHIPS, INC., Bedford, MA (US)

(21) Appl. No.: 12/239,224

(22) Filed: Sep. 26, 2008

Related U.S. Application Data
(60) Provisional application No. 60/975,504, filed on Sep. 26, 2007.

(51) Int. Cl. A61L 27/54

(52) U.S. Cl. 424/423

(57) ABSTRACT

Implantable drug delivery devices are provided which are adapted to be installed adjacent to at least a portion of an orthopedic prosthetic device implanted on a surgically prepared bone section of a patient. For example, the prosthetic device may be a hip joint replacement or knee joint replacement. The drug delivery device may include a body having a core formed from a non-biodegradable material formed in the shape of a sleeve and having an inner surface and an outwardly directed outer surface, wherein the outer surface has a drug formulation for release thereof into the body of the animal following implantation of the prosthetic device within the body of the animal. The outer surface may define one or more pockets for holding the drug formulation.

### CROSS-REFERENCE TO RELATED APPLICATIONS

This application claims the benefit of U.S. Provisional Application No. 60/975,504, filed Sep. 26, 2007. The application is incorporated herein by reference in its entirety.

### BACKGROUND OF THE INVENTION

This invention is generally in the field of implantable medical devices and methods for patient therapy and prophylaxis.

Implantable medical devices for the delivery of drugs to human or animal patients over extended periods are known. For example, U.S. Pat. No. 5,797,898, U.S. Pat. No. 6,527,762, U.S. Pat. No. 6,491,666, and U.S. Pat. No. 6,976,982, to Santini Jr. et al., describe devices for the storage and release of drug formulations from multi-reservoir devices.

It also known to have a bioabsorbable plug, containing a therapeutic agent, that is threaded to or inserted into a recess in a prosthesis, or is inserted into an implantable mesh bag that is sutured to soft tissue or a bone fastener, as in U.S. Pat. No. 6,916,483 to Ralph et al.

It is also known to deliver a drug, such as an antibiotic, in conjunction with surgical implantation of various prosthetic devices, such as orthopedic or dental devices. For example, it is known to include an antibiotic in bone cement used to secure an orthopedic implant. See Jiranek, et al., “Antibiotic-Loaded Bone Cement for Infection Prophylaxis in Total Joint Replacement,” J. Bone Joint Surg. Am. 88:2487-500 (2006). U.S. Pat. No. 6,916,483 to Ralph et al.; U.S. Patent Application Publication No. 2007/0088442 to Cima et al.; U.S. Patent Application Publication No. 2007/0016163 to Santini Jr. et al.; and U.S. Patent Application Publication No. 2006/0093646 to Cima et al. describe devices for the release of a drug from an orthopedic or dental implant device. U.S. Patent Application Publication No. 2006/0093646 also discloses at paragraph [0068] a flexible drug delivery device, formed of a polymeric film containing pellets of a therapeutic agent, that is wrapped around bone tissue.

It would be desirable to provide improved or alternative techniques and devices for controlled local delivery of one or more therapeutic or prophylactic agents to the bodily tissues surrounding a site of implantation of a prosthetic implant device. These techniques and devices desirably would provide well-controlled delivery of the drug(s) over an extended period, such as several days, weeks, or months. The drug delivery devices desirably would be relatively simple to implant during the prosthetic implantation surgery, would not interfere with operation of the prosthetic device, and would not require explanation following completion of drug delivery.

### SUMMARY OF THE INVENTION

In one aspect, implantable drug delivery devices are provided which are adapted to be installed adjacent to at least a portion of an orthopedic prosthetic device implanted on a surgically prepared bone section of a patient. The drug delivery device may include a body having a core formed from a non-biodegradable material formed in the shape of a sleeve and having an inner surface and an outwardly directed outer surface, wherein the outer surface has a drug formulation for release thereof into the body of the animal following implantation of the prosthetic device within the body of the patient. The body of the drug delivery device is shaped for association with at least a portion of one or both of the prosthetic device and the surgically prepared bone section. The outer surface may define at least one pocket for holding the drug formulation.

In one embodiment of the drug delivery device, the body has a radially inwardly directed inner surface, and a radially outwardly directed outer surface, and the inner surface defines an aperture that extends though the device body. The body may define an open shape having two free ends. The body of the drug delivery holds a drug formulation for in vivo release through the outer surface following implantation of the prosthetic device within the patient and installation of the drug delivery device.

In one particular embodiment, the prosthetic device is a hip replacement joint having a femoral portion including a hip stem having a neck, wherein the body of the drug delivery device is adapted to receive a portion of said hip stem in confronting relation around three sides of the hip stem or to receive the hip stem with the inner surface of the body in surrounding relation to the neck. In another particular embodiment, the prosthetic device is a knee replacement joint having an articular surface, wherein the body is adapted to engage a peripheral portion of the articular surface and the body in the shape of a sleeve has an approximate C-shaped extent, portions of the body being smooth in regions to be overlaid by muscle or tendon tissue.

In another aspect, a method is provided for delivering a drug to a human or other mammalian animal patient, in which an orthopedic prosthetic device is implanted. The method includes implanting the orthopedic prosthetic device onto a surgically prepared bone section of the human or other animal, and surrounding at least a portion of the prosthetic device with an implantable drug delivery device. Preferably, the combination is adapted to prevent relative motion between the prosthetic device and the drug delivery device.

### BRIEF DESCRIPTION OF THE DRAWINGS

FIG. 1 is a perspective view of an embodiment of a drug delivery device that is adapted for use with a standard knee prosthesis.

FIG. 2 is a top view of the embodiment of the drug delivery device shown in FIG. 1.

FIG. 3 is a front view of the embodiment of the drug delivery device shown in FIG. 1.

FIG. 4 is a back view of the embodiment of the drug delivery device shown in FIG. 1.

FIG. 5 is a left side view of the embodiment of the drug delivery device shown in FIG. 1.

FIG. 6 is a front perspective view of the embodiment of the drug delivery device shown in FIG. 1, illustrating the device implanted adjacent to an implanted knee prosthesis, with a front portion of the device positioned behind the patellar tendon and a left portion of the device positioned behind the lateral collateral ligament.

FIG. 7 is a front perspective view of the drug delivery device implanted as shown in FIG. 6, illustrating the front portion of the device adjacent to the articular surface of the knee prosthesis with the patella and the patellar tendon removed.

FIG. 8 is a left rear perspective view of the drug delivery device implanted as shown in FIG. 6, illustrating the left portion of the device positioned behind the lateral collateral ligament.

FIG. 9 is a right side view of the drug delivery device implanted as shown in FIG. 6, illustrating the right portion of the device positioned beneath the medial collateral ligament.

FIG. 10 is another top view of the embodiment of the drug delivery device shown in FIG. 1.

FIG. 11 is a cross-sectional view of the embodiment of the drug delivery device taken along line 11-11 in FIG. 10.

FIG. 12 is a rear perspective view of the embodiment of the drug delivery device taken along line 12-12 in FIG. 11.

FIG. 13 is another side view of the embodiment of the drug delivery device shown in FIG. 1.

FIG. 14 is a cross-sectional view of the embodiment of the drug delivery device taken along line 14-14 in FIG. 13.

FIG. 15 is a cross-sectional view of the embodiment of the drug delivery device taken along line 15-15 in FIG. 13.

FIG. 16 is another perspective view of an embodiment of the drug delivery device shown in FIG. 1.

FIG. 17 is a front view of the embodiment of the drug delivery device shown in FIG. 16.

FIG. 18 is a rear view of the embodiment of the drug delivery device shown in FIG. 16.

FIG. 19 is a top view of the embodiment of the drug delivery device shown in FIG. 16.

FIG. 20 is a bottom view of the embodiment of the drug delivery device shown in FIG. 16.

FIG. 21 is a left side view of the embodiment of the drug delivery device shown in FIG. 16.

FIG. 22 is an upper perspective view of an embodiment of a drug delivery device adapted for use with a standard hip prosthesis.

FIG. 23 is a lower perspective view of the embodiment of the drug delivery device shown in FIG. 22.

FIG. 24 is a perspective view of the embodiment of the drug delivery device shown in FIG. 22, illustrating the device implanted adjacent to a portion of an implanted hip prosthesis.

FIG. 25 is another perspective view of the embodiment of the drug delivery device shown in FIG. 22, illustrating the device positioned about a portion of an implanted hip prosthesis with a head and an acetabular cup of the hip prosthesis removed so that a neck of the hip prosthesis is visible.

FIG. 26 is a side view of the embodiment of the drug delivery device shown in FIG. 22, illustrating the device implanted adjacent to a portion of an implanted hip prosthesis with a head and an acetabular cup of the hip prosthesis removed.

FIG. 27 is a side view of the embodiment of the drug delivery device shown in FIG. 22, illustrating the device implanted adjacent to a portion of an implanted hip prosthesis with a head and an acetabular cup of the hip prosthesis removed.

FIG. 28 is a front view of the embodiment of the drug delivery device shown in FIG. 22, illustrating the device implanted adjacent to a portion of an implanted hip prosthesis, with an acetabular cup in a non-rotated position.

FIG. 29 is a another front view of the embodiment of the drug delivery device shown in FIG. 22, illustrating the device implanted adjacent to a portion of an implanted hip prosthesis, with an acetabular cup in a rotated position.

FIG. 30 is a top view of the embodiment of the drug delivery device shown in FIG. 22, illustrating the device implanted adjacent to a portion of an implanted hip prosthesis with a head and an acetabular cup of the hip prosthesis removed.

FIG. 31 is an upper perspective view of another embodiment of a drug delivery device adapted for use with a standard hip prosthesis.

FIG. 32 is a lower perspective view of the embodiment of the drug delivery device shown in FIG. 31.

FIG. 33 is a perspective view of the embodiment of the drug delivery device shown in FIG. 31, illustrating the device positioned about a portion of an implanted hip prosthesis with a head and an acetabular cup of the hip prosthesis removed so that a neck of the hip prosthesis is visible.

FIG. 34 is a side view of the embodiment of the drug delivery device shown in FIG. 31, illustrating the device implanted adjacent to a portion of an implanted hip prosthesis with a head and an acetabular cup of the hip prosthesis removed.

FIG. 35 is a top view of the embodiment of the drug delivery device shown in FIG. 31, illustrating the device implanted adjacent to a portion of an implanted hip prosthesis with a head and an acetabular cup of the hip prosthesis removed.

FIG. 36 is another perspective view of the embodiment of the drug delivery device shown in FIG. 31, illustrating a set screw maintaining a position of the device against a hip stem of the implanted hip prosthesis.

FIG. 37 is a side view of the embodiment of the drug delivery device shown in FIG. 31, further illustrating the set screw maintaining the position of the device against the hip stem of the implanted hip prosthesis.

FIGS. 38-41 are additional perspective, top, side, and bottom views of the embodiment of the drug delivery device shown in FIG. 31.

FIG. 42 is a side view of the embodiment of the drug delivery device taken along perspective 42-42 in FIG. 38.

FIG. 43 is a cross-sectional view of the embodiment of the drug delivery device taken along line 43-43 in FIG. 40.

FIG. 44 is a cross-sectional view of the embodiment of the drug delivery device taken along line 44-44 in FIG. 40.

FIG. 45 is a view of the embodiment of the drug delivery device taken along line 45-45 in FIG. 44.

FIG. 46 is a view of the embodiment of the drug delivery device taken along line 46-46 in FIG. 45.

FIG. 47 is a rear view of the embodiment of the drug delivery device shown in FIG. 38.

FIG. 48 is a view of the embodiment of the drug delivery device taken along line 48-48 in FIG. 47.

FIG. 49 is a perspective view of an embodiment of a drug delivery device formed in the shape of a closed sleeve or cap.

FIG. 50 is a perspective view of the embodiment of the drug delivery device shown in FIG. 49, illustrating the device positioned about an upper portion of a hip implant.

FIG. 51 is a side view of the embodiment of the drug delivery device shown in FIG. 49, illustrating the device positioned about an upper portion of a hip implant.

FIG. 52 is a top perspective view of the embodiment of the drug delivery device shown in FIG. 49, illustrating the device positioned about an upper portion of a hip implant.

FIG. 53 is a perspective cross-sectional view of the embodiment of the drug delivery device shown in FIG. 49, illustrating protruding flat areas that assist in securing the device to the hip implant, and corresponding relief areas.

FIG. 54 is a front cross-sectional view of the embodiment of the drug delivery device shown in FIG. 49, taken through the relief areas.

FIG. 55 is a perspective cross-sectional view of the embodiment of the drug delivery device shown in FIG. 49, taken through the protruding flat areas.

FIG. 56 is a perspective cross-sectional view of another embodiment a drug delivery device, which is adapted to be secured to the hip implant with cement.

FIG. 57 is a cross-sectional view of another embodiment of a drug delivery device, which is molded to mate with a tapered hip stem.

FIG. 58 is a cross-sectional view of an embodiment of a tapered hip stem, which includes multiple discrete reservoirs for holding a drug formulation.

### DETAILED DESCRIPTION OF THE INVENTION

Implantable drug delivery devices have been developed for use in combination with an implantable prosthetic device, such as in a partial or complete joint replacement. For example, the drug delivery device may be used with various hip or knee replacement surgeries, for example for infection prophylaxis. The devices advantageously provide controlled, local delivery of antibiotic agents and/or other drugs to the site of implantation of the prosthetic implant. For example, the device may serve as a universal drug delivery device that can easily be incorporated into total hip arthoplasty (THA) or a total knee arthroplasty (TKA) to provide precise local dosing and pharmacokinetics from formulations optimized for stability, while avoiding late elution.

The devices advantageously can provide controlled delivery of known quantities of antibiotics or other drugs, more precisely than from a cement, such as in a procedure where the prosthesis is secured using a drug-containing bone cement, the use of which may limit the selection or doses of drug that can be used without comprising cement strength. In addition, the present device can provide drug delivery with implant procedures that are cementless.

The devices beneficially can be used with various commercially-available or other prosthetic implant devices and generally do not require modification of the prosthetic device design in order to provide the drug delivery functionality. Advantageously, the devices may be directly secured primarily or partially to the implanted joint prosthetic device, and not exclusively secured to patient tissues at the site of implantation.

In a preferred embodiment, the devices release the drug from discrete reservoirs (also referred to as “pockets”) defined in the body, rather than from a monolithic device body, which confers several advantages including decoupling the mechanical properties of the device from the drug formulation, easing delivery of multiple drugs (without concern of drug incompatibilities in a single formulation), and protecting/stabilizing of the drug formulation in the device.

### The Devices

In one aspect, an implantable drug delivery device is provided which is adapted to be installed at or adjacent an implantable orthopedic prosthetic device inserted onto a surgically prepared bone section of an animal, such as a human or other mammalian patient. In one embodiment, the implantable drug delivery device includes a body formed in the shape of a sleeve and having an inner surface and an outwardly directed outer surface, the outer surface having a therapeutic agent for release thereof into the body of the animal following implantation of the prosthetic device within the body of the animal.

In one embodiment, the sleeve defines an open shape having free ends. In another embodiment, the sleeve defines a closed annular shape. In various embodiments, the body of the device may be formed of a biocompatible metal, polymer, or ceramic. For example, the body may be made of an alloy of titanium such as titanium-aluminum-vanadium (such as Ti-6A1-4V), a stainless steel, a cobalt chrome alloy, zirconia, or a combination thereof. In another embodiment, the body may be formed of a non-degradable polymer, such as a cross-linked or non-cross-linked polyethylene, and is preferably rigid. In another embodiment, the body may be formed of a degradable polymer, such as poly(lactic-co-glycolic acid) (PLGA), poly(lactic acid)s, poly(glycolic acid)s, or degradable poly(anhydride-co-imides), a polytetrafluoroethylene, a polyester, a silicone, or a combination thereof. The body may be a composite or multilayer structure. The degradable polymer may be a drug-eluting polymeric material.

In one embodiment, the device body is formed of a drug-eluting polymer. The drug may be loaded in the polymer, such as homogeneously dispersed throughout a polymeric matrix material or heterogeneously distributed throughout the matrix material.

In one embodiment, the outer surface of the body defines one or more pockets, i.e., reservoirs, for holding at least one therapeutic agent. For example, an array of several discrete reservoirs may be provided in/on one or more surface regions of the implant device. The number of reservoirs and the size of the reservoirs may vary depending on, for example, the “real estate” available on or in the implant device for holding the reservoirs and/or the total volume of drug (or drug formulation) to be implanted in and delivered to the patient. For example, the total volume of the reservoirs in a single device may be between about 0.5 mL and about 5 mL, e.g., about 2 mL or about 3 mL. A reservoir may extend substantially into or through the body structure. The shape and dimensions of the reservoir, along with the number and size of the reservoir openings in the surface of the device body, can be selected to influence the rate of drug diffusion from a reservoir.

In various embodiments, the reservoirs are discrete, non-deformable, and disposed in an array in the body. The reservoir openings may be along one or more surface regions of the device body, and may be provided around surfaces to release drug in multiple directions. The body may include tens or hundreds of reservoirs arrayed across the exterior surface. The body may be substantially rigid and the reservoirs may be non-deformable.

In one embodiment, the reservoirs are macroreservoirs. A “macroreservoir” is a reservoir suitable for storing and releasing/exposing a quantity of material larger than a microquantity. The macroreservoir has a volume greater than 500 μL (e.g., greater than 600 μL, greater than 750 μL, greater than 900 μL, greater than 1 mL, etc.) and less than 10 mL (e.g., less than 5 mL, less than 3 mL, less than 2 mL, less than 1 mL, etc.). In one embodiment, the reservoirs are microreservoirs. A “microreservoir” is a reservoir suitable for storing and releasing/exposing a microquantity of material, such as a drug formulation. The term “microquantity” refers to volumes from 1 mL up to 500 μL. In one embodiment, the microreservoir has a volume equal to or less than 500 μL (e.g., less than 250 μL, less than 100 μL, less than 50 μL, less than 25 μL, less than 10 μL, etc.) and greater than about 1 nL (e.g., greater than 5 nL, greater than 10 nL, greater than about 25 nL, greater than about 50 nL, greater than about 1 μL, etc.). In one embodiment, the microquantity is between 1 nL and 1 μL. In another embodiment, the microquantity is between 10 nL and 500 nL. In still another embodiment, the microquantity is between about 1 μL and 500 μL. Unless explicitly indicated to be limited to either micro- or macro-scale volumes/quantities, the term “reservoir” is intended to encompass both.

In a preferred embodiment, the reservoirs and drug formulation are designed such that the formulation, which typically is in a solid form, cannot be released from the reservoir as a whole (e.g., as a pellet) but rather can only be released in a gradual, top down fashion as the drug formulation dissolves/diffuses. This may be controlled, for example, by the shape of the reservoir (e.g., a reservoir opening narrower than the distal periphery of the drug formulation pellet), the interior surface of the reservoir (e.g., a rough surface), the use of sticky or viscous excipient materials with the drug, the use of a flexible liner material lining the reservoir interior, and/or the use of a relatively flexible body material (e.g., silicone, polymer).

In an alternative embodiment, the device body, or a portion thereof such as the outer surface, is formed of a drug-eluting polymer and has no discrete drug-containing reservoirs located about the surface of the device, such that the drug is loaded in the polymer, e.g., homogeneously dispersed throughout a polymeric matrix material or heterogeneously distributed throughout the matrix material.

As used herein, the terms “drug” and “therapeutic agent” are used interchangeably. These terms include any therapeutic or prophylactic active pharmaceutical ingredient (API). A variety of different kinds of drugs may be released from the implant device. The drug may be one that is useful for pain management, infection control, osteointegration, thrombophrophylaxsis, oncology, or a combination thereof. In a preferred embodiment, the therapeutic agent is an antibiotic, such as gentamicin, vancomycin, erythromycin, tobramycin, cefazolin, ciprofloxacin, clindamycin, ticarcillin, cefuroxime, cefotaxime, fusidic acid, or a combination thereof. Other aminoglycoside or cephalosporin antibiotics may also be used in infection prophylaxis. In one example, the device is used to deliver one or more antibiotics for at least 30 days at therapeutically effective levels. In another example, the device is used to deliver one or more antibiotics for up to 3 months at therapeutically effective levels.

In one embodiment, multiple antibiotics may be delivered from different reservoirs for a synergistic effect, potentially at lower doses per antibiotic.

In one embodiment, the device includes a plurality of pockets, where a subset of the pockets has a different formulation than that of another subset of the pockets. For example, different pockets may have different drugs or different formulations of the same drug. The pockets may be tailored to provide different kinetics of release. This may be accomplished by providing the drug in the form of multiple discrete layers of drug and a non-bioactive, release controlling substance such as a biodegradable polymer, or by providing the drug in the form of a gradient composition, in order to provide staged, controlled release.

As used herein, the term “drug formulation” refers to a composition that comprises a drug. The drug formulation may include one or more pharmaceutically acceptable excipients, which are known in the art.

The drug formulation may be in essentially any form, such as a pure solid or liquid, a gel or hydrogel, a solution, an emulsion, a slurry, or a suspension. In a preferred embodiment, the drug formulation is in a monolithic, dry solid form, or in the form of a collection of particles (e.g., microparticles or nanoparticles), particularly for purposes of maintaining or extending the stability of the drug over a commercially and medically useful time, such as during storage in a drug delivery device until the drug needs to be administered. These solid forms may be provided by lyophilization of a drug solution or suspension directly in the reservoirs. Alternatively, prefabricated pellets that are approximately the size of the individual reservoirs may be formed outside the reservoirs (e.g., in a mold) and then may be transferred and deposited into the reservoirs, such as by a pick-and-place technique. Alternatively, the drug formulation may be in a gel, liquid, or suspension form. The particular formulation in the reservoirs of a single device may be the same as or different from one another across a plurality of the reservoirs.

The drug formulation may include a drug in combination with other materials to control or enhance the rate and/or time of release from an opened reservoir. In various embodiments, the drug formulation further includes one or more matrix materials. In one example, the matrix material comprises one or more synthetic polymers. Exemplary materials include synthetic polymers, such as PLGA, PEG, and poly-L-lactide (PLLA), and/or naturally occurring polymers such as hyaluronic acid, chitosan, and alginate. The naturally occurring polymers may or may not be crosslinked by methods known to the art. In another example, the one or more matrix materials may comprise a biodegradable, bioerodible, water-soluble, or water-swellable matrix material. In one embodiment, the drug is distributed in the matrix material and the matrix material degrades or dissolves in vivo to controllably release the therapeutic or prophylactic agent. The drug may be heterogeneously distributed in the reservoir or may be homogeneously distributed in the reservoir. The matrix material can be a “release system,” as described in U.S. Pat. No. 5,797,898, the degradation, dissolution, or diffusion properties of which can provide a method for controlling the release rate of the drug molecules.

Representative examples of other drugs that may be useful with the present devices include anti-thrombotic agents, anti-inflammatories, vaccines, vectors for gene therapy, polypeptides, nucleic acids (DNA, siRNA), interferons, antibodies, hormones, and chemotherapeutic agents. In various embodiments, the drug can be selected from amino acids, vaccines, antiviral agents, gene delivery vectors, interleukin inhibitors, immunomodulators, neurotropic factors, neuroprotective agents, antineoplastic agents, chemotherapeutic agents, polysaccharides, anti-coagulants (e.g., LMWH, pentasaccharides), antibiotics (e.g., immunosuppressants), analgesic agents, and vitamins. The drug may be a protein, such as glycoproteins, enzymes (e.g., proteolytic enzymes), hormones or other analogs (e.g., LHRH, steroids, corticosteroids, growth factors), antibodies (e.g., anti-VEGF antibodies, tumor necrosis factor inhibitors), cytokines (e.g., α-, β-, or γ-interferons), interleukins (e.g., IL-2, IL-10), and diabetes/obesity-related therapeutics (e.g., insulin, exenatide, PYY, GLP-1 and its analogs). The drug may be a gonadotropin-releasing hormone (LHRH) analog, such as leuprolide. The drug may comprise a parathyroid hormone, such as a human parathyroid hormone or its analogs, e.g., hPTH(1-84) or hPTH(1-34). The drug may be selected from nucleosides, nucleotides, and analogs and conjugates thereof. The drug may comprise a peptide with natriuretic activity. The drug may be selected from diuretics, vasodilators, inotropic agents, anti-arrhythmic agents, Ca⁺ channel blocking agents, anti-adrenergics/sympatholytics, and renin angiotensin system antagonists. The drug may be a VEGF inhibitor, VEGF antibody, VEGF antibody fragment, or another anti-angiogenic agent. The drug may be a prostaglandin, a prostacyclin, or another drug effective in the treatment of peripheral vascular disease. The drug may be an angiogenic agent, such as VEGF. The drug may be an anti-inflammatory, such as dexamethasone. A single device may include a single drug or a combination of two or more drugs.

The release of drug from a single reservoir may be tailored to provide a temporally modulated release profile (e.g., pulsatile release), such as when time variation in plasma levels is desired, or a more continuous or consistent release profile, such as when constant plasma levels are needed to enhance a therapeutic effect, for example. Pulsatile release can be achieved from an individual reservoir, from a plurality of reservoirs, or a combination thereof. For example, where each reservoir provides only a single pulse, multiple pulses (i.e., pulsatile release) are achieved by temporally staggering the single pulse release from each of several reservoirs. Alternatively, multiple pulses can be achieved from a single reservoir by incorporating several layers of a release system and other materials into a single reservoir. Continuous release can be achieved by incorporating a release system that degrades, dissolves, or allows diffusion of molecules through it over an extended period. In addition, continuous release can be approximated by releasing several pulses of molecules in rapid succession (“digital” release). In one embodiment, the drug formulation within a reservoir comprises layers of a drug or drugs and a non-drug material, wherein the multiple layers provide pulsed drug release due to the intervening layers of non-drug. Such a strategy can be used to obtain complex release profiles.

In one embodiment, the body further includes a fixation means for securing the device to the prosthetic device, the bone tissue, or both. For example, the fixation means may be chosen from the group of fixation means consisting of: a hole adapted to receive a screw, a pin, a suture, a cement, or a glue; a detent adapted to engage a groove; an indentation adapted to receive a cement or glue; a snap fitting; a pressure fitting; a screw fitting; a screw; a pin; a suture; a cement; a glue; and a groove. Bone cements are well known in the art, and for example, may be a polymethylmethacrylate. The fixation means enables the surgeon to secure the device at the desired site once placed in adjacent to the surgically prepared bone section and/or the implant.

In one embodiment, the prosthetic device is a portion of a hip or knee replacement joint. In one particular example, the prosthetic device is a hip replacement joint having a femoral portion including a hip stem having a neck, wherein in the drug delivery device (i) a portion of the sleeve is adapted to be cemented to at least a portion of the hip stem or the surgically prepared bone section, and (ii) the aperture is adapted to receive a portion of the hip stem in confronting relation around three sides of the hip stem. In another example, the prosthetic device is a hip replacement joint having a femoral portion including a hip stem having a neck, wherein in the drug delivery device the sleeve is adapted to receive the hip stem with the inner surface in surrounding relation to the neck.

In yet another example, the prosthetic device is a knee replacement joint having an articular surface, and the drug delivery device the sleeve defines an open shape having free ends, wherein (i) the sleeve is adapted to engage a peripheral portion of the articular surface; and (ii) the sleeve has an approximate C-shaped extent, portions of the sleeve being smooth in regions to be overlaid by muscle, tendon, or ligament tissue.

In another aspect, an implantable drug delivery device is provided which is adapted to be installed at or adjacent an implantable orthopedic prosthetic device inserted onto a surgically prepared bone section of an animal. The device includes a body having a radially inwardly directed inner surface defining an aperture, and a radially outwardly directed outer surface; said body having a thickness dimension defined in a generally radial direction extending from said inner surface to said outer surface; wherein said aperture extends over the length of the thickness dimension; and wherein said outer surface holds a therapeutic agent for release thereof into the body of the animal following implantation of the prosthetic device within the body of the animal.

In one embodiment of this drug delivery device, the prosthetic device is a portion of a hip or knee replacement joint. The prosthetic device may be a hip replacement joint having a femoral portion including a hip stem having a neck, wherein (i) said body is adapted to be cemented to at least a portion of said hip stem or said surgically prepared bone section, and (ii) said aperture is adapted to receive a portion of said hip stem in confronting relation around three sides of said hip stem.

In another aspect, an implantable drug delivery device is provided which is adapted to be installed on an implantable orthopedic prosthetic device inserted onto a surgically prepared bone section of an animal, wherein the device includes a body having a first wall and an outer surface, said first wall adapted to be cemented to at least one of a portion of said implantable prosthetic device or said surgically prepared bone section; an aperture formed in said body and extending through said body, said aperture adapted to receive a portion of said implantable prosthetic device; and said outer surface holds a therapeutic agent for release thereof into the body of the animal following implantation of the prosthetic device within the body of the animal.

The outer surface may define at least one pocket for holding said therapeutic agent, which may be for example an antibiotic. The body may further include a fixation means chosen from the group of fixation means consisting of one or more holes, pins, detents, and indentations for receiving a cement.

The prosthetic device may be a portion of a hip or knee replacement joint. In one embodiment, the prosthetic device is a hip replacement joint having a femoral portion including a hip stem having a neck, wherein (i) said first wall is adapted to be cemented to at least a portion of said hip stem or said surgically prepared bone section, and (ii) said aperture is adapted to receive a portion of said hip stem in confronting relation around three sides of said hip stem.

Reservoir openings optionally may be closed off by at least one reservoir cap, membrane, film, or other structure. For example, each reservoir may further include a discrete reservoir cap. A reservoir cap may cover the opening(s) of the reservoir to protect the drug formulation until it is desired to initiate release or the reservoir cap may serve as a diffusion limiting, i.e., release rate controlling, material. A reservoir cap is a thin film or other structure suitable for separating the contents of a reservoir from the environment outside of the reservoir. The reservoir caps are formed from a material or mixture of materials that degrade, dissolve, or otherwise disintegrate in vivo, or that do not degrade dissolve, or disintegrate, but become permeable in vivo to the drug molecules.

The device body or reservoir cap may include a wrap or coating of a semi-permeable or degradable material, such as a polymer, to control transport of molecules into or out of the reservoir when the device is in vivo. The reservoir caps for each of the reservoirs are designed to open at specified times, thereby delivering the reservoir content sequentially at pre-specified times. These reservoir caps may be independently disintegrated or permeabilized or groups of the reservoir caps can be actuated simultaneously. This reservoir opening may be passively controlled through the disintegration of the reservoir cap. In a passive control system for example, the timing can be controlled by selecting the reservoir cap dimension, composition, and structure. Simultaneous actuation of the reservoir contents can be obtained by covering multiple reservoirs with reservoir caps of identical dimension, composition, and structure that will release the contents of different reservoirs at the same time.

The compositions of the reservoir caps may be selected from materials that will disintegrate in response to an environment existing in vivo in the patient or in response to a component contained in the reservoir. Examples of environmental conditions include, but are not limited to temperature, water, an electrolyte, and enzymes. Alternatively, the material of the reservoir cap may also be selected so that it will disintegrate when exposed to a form of energy that is applied to the patient from an external source or implanted internal source, such as acoustic (audible or ultrasonic) energy, magnetic energy, electromagnetic radiation (e.g., UV, visible, IR light, RF energy, or X-ray).

As used herein, the term “disintegrate” refers to degrading, dissolving, rupturing, fracturing or some other form of mechanical failure, as well as fracture and/or loss of structural integrity of the reservoir cap due to a chemical reaction or phase change (e.g., melting or transitioning from a solid to a gel), unless a specific one of these mechanisms is indicated. Hydrolytic decomposition is a preferred form of disintegration.

In preferred embodiments, the reservoir caps are selected to dissolve or biodegrade in vivo, without any intervention by the patient or caregiver. In one particular embodiment, the reservoir caps are formed of a biocompatible polymer, such as a poly(lactic acid), poly(glycolic acid), or poly(lactic-co-glycolic acid)s, as well as degradable poly(anhydride-co-imides), of a composition and thickness designed to disintegrate by hydrolysis in a prescribed timeframe, releasing the contents of the reservoir.

As used herein, the term “permeabilize” is used broadly to include without limitation some form of physical change that does not alter the chemical composition or dry mass of the membrane but changes the capability of the membrane to contain the reservoir contents. In a preferred embodiment of permeabilization, the polymer swells, changing its porosity without decomposition so that the reservoir contents are in contact with fluid external to the device and are releasable through the resulting open pore structure.

In a preferred embodiment, a discrete reservoir cap completely covers one of the reservoir's openings. In another embodiment, a discrete reservoir cap covers two or more, but less than all, of the reservoir's openings.

Representative examples of reservoir cap materials include polymeric materials and various types of semi-permeable membranes, and non-polymeric materials. In a preferred embodiment, the reservoir caps are non-porous and are formed of a bioerodible or biodegradable material, known in the art, such as a synthetic polymer, e.g., a polyester (such as PLGA), a poly(anhydride), or a polycaprolactone. The reservoir cap may be a multilayer structure. For example an inner layer may be porous or otherwise control diffusion once the outer, non-permeable layer has disintegrated. The reservoir caps of a single device may be made of different materials, may have different thicknesses, may have different degrees of cross-linking, or a combination thereof, for the purpose of opening different reservoirs at different times relative to one another.

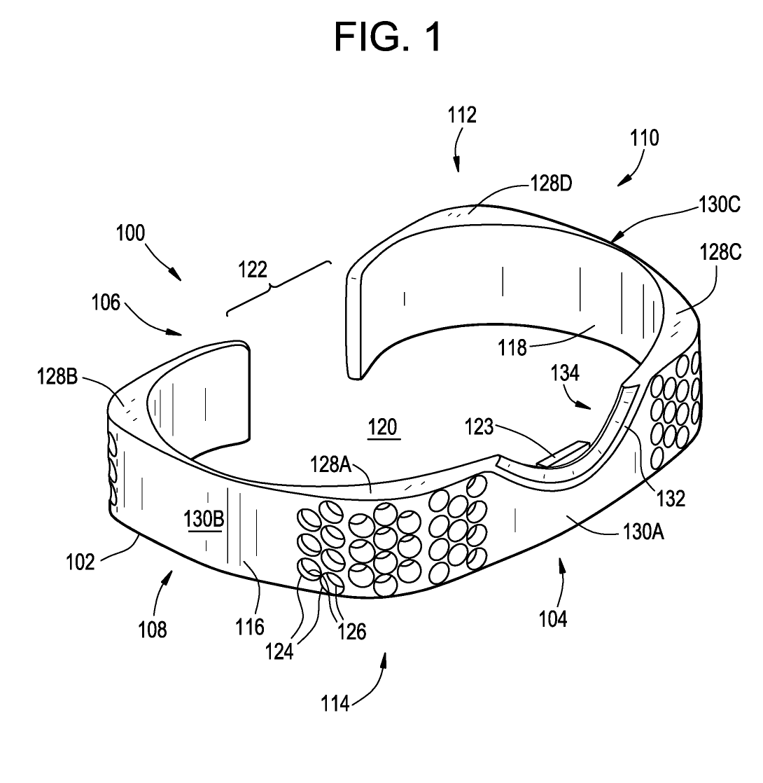

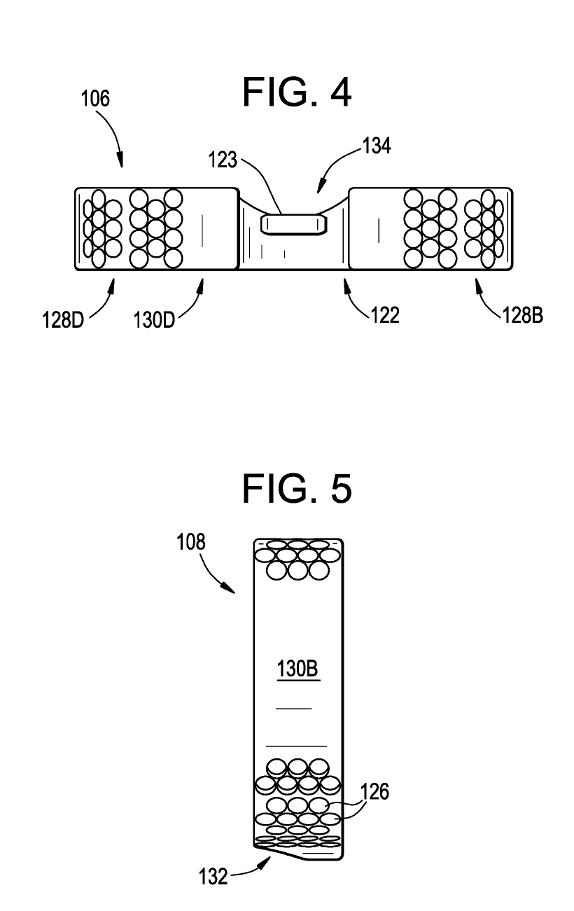

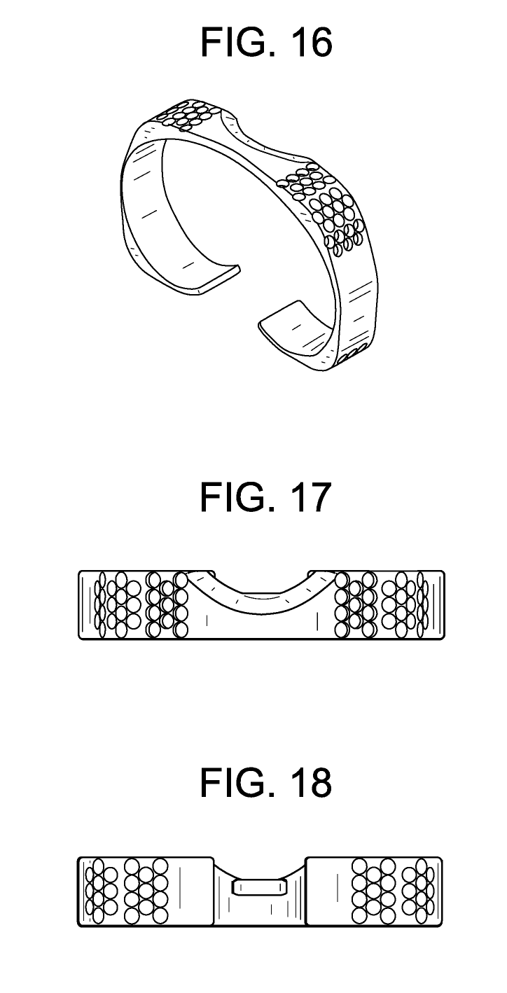

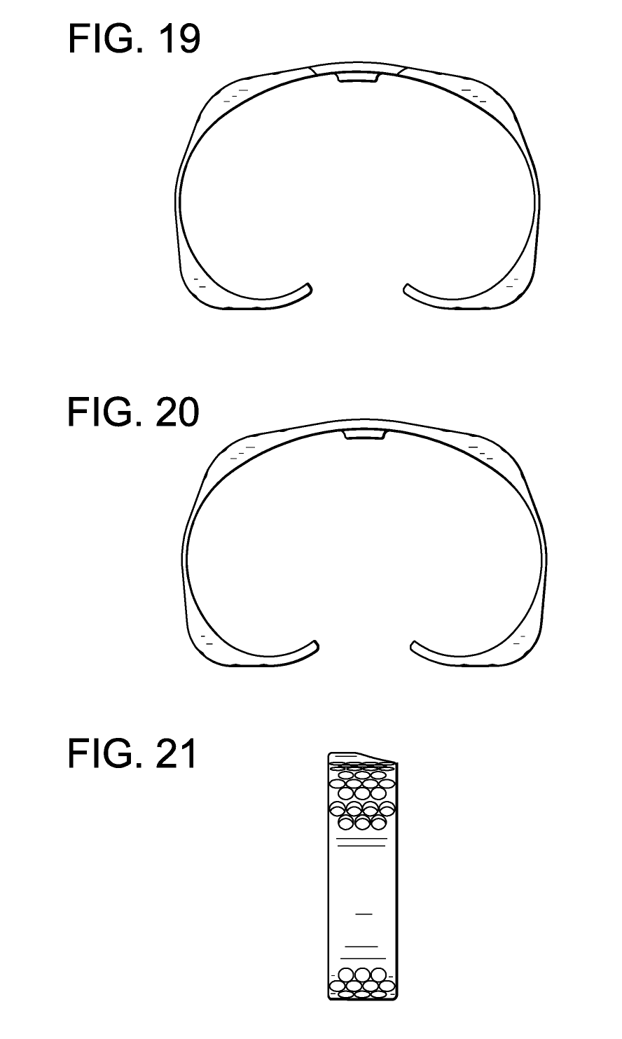

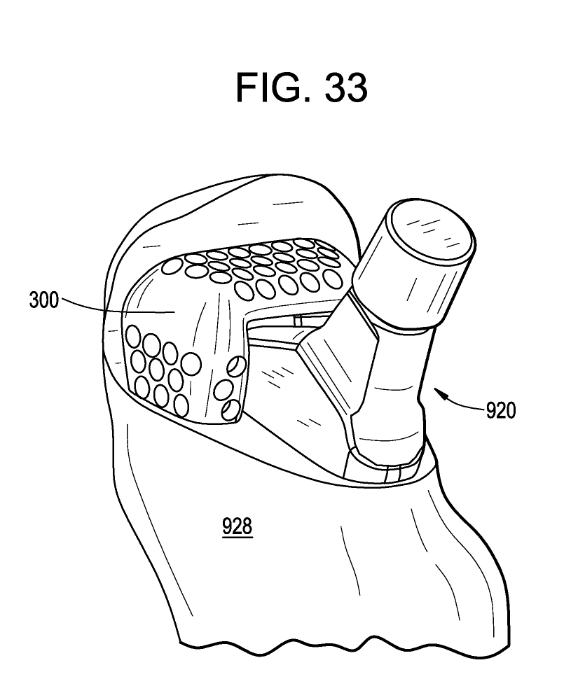

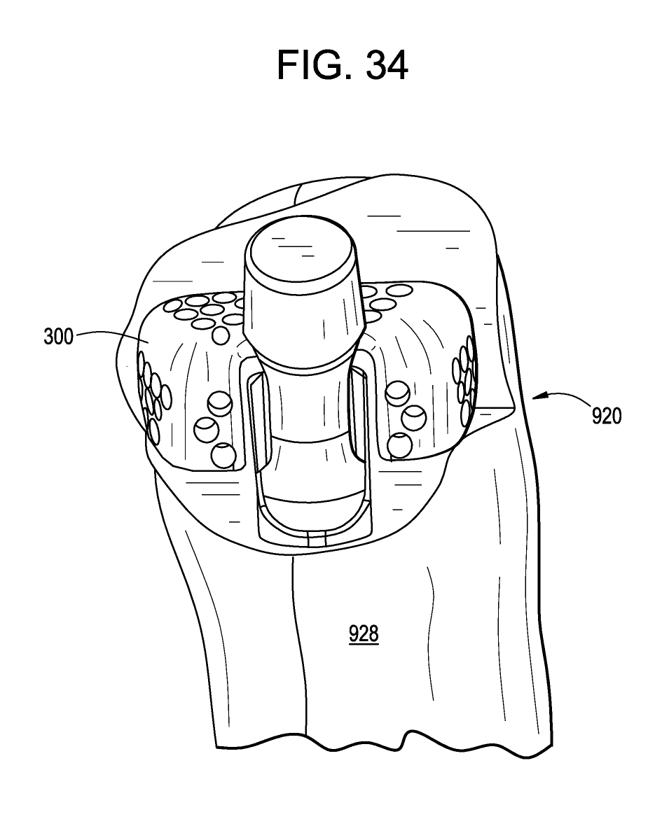

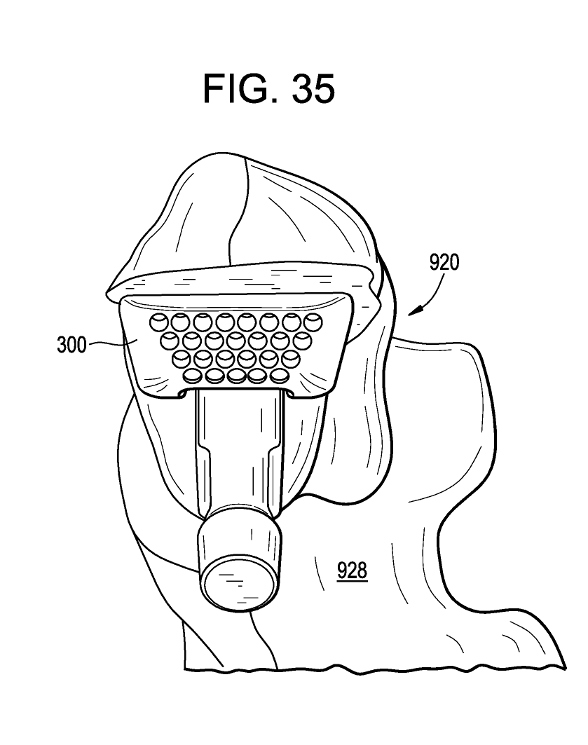

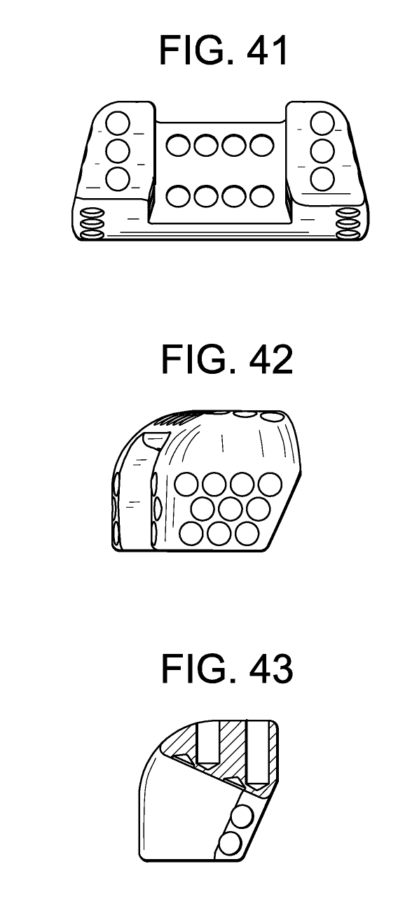

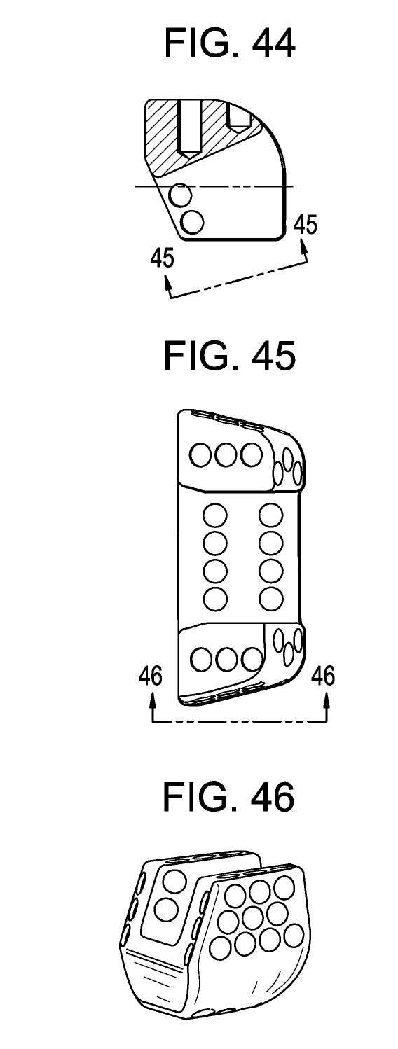

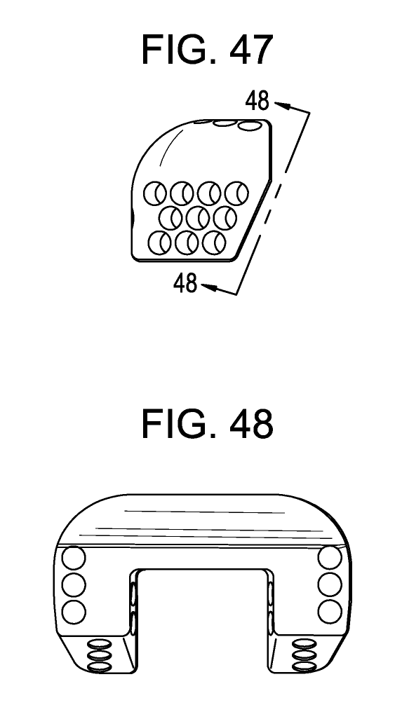

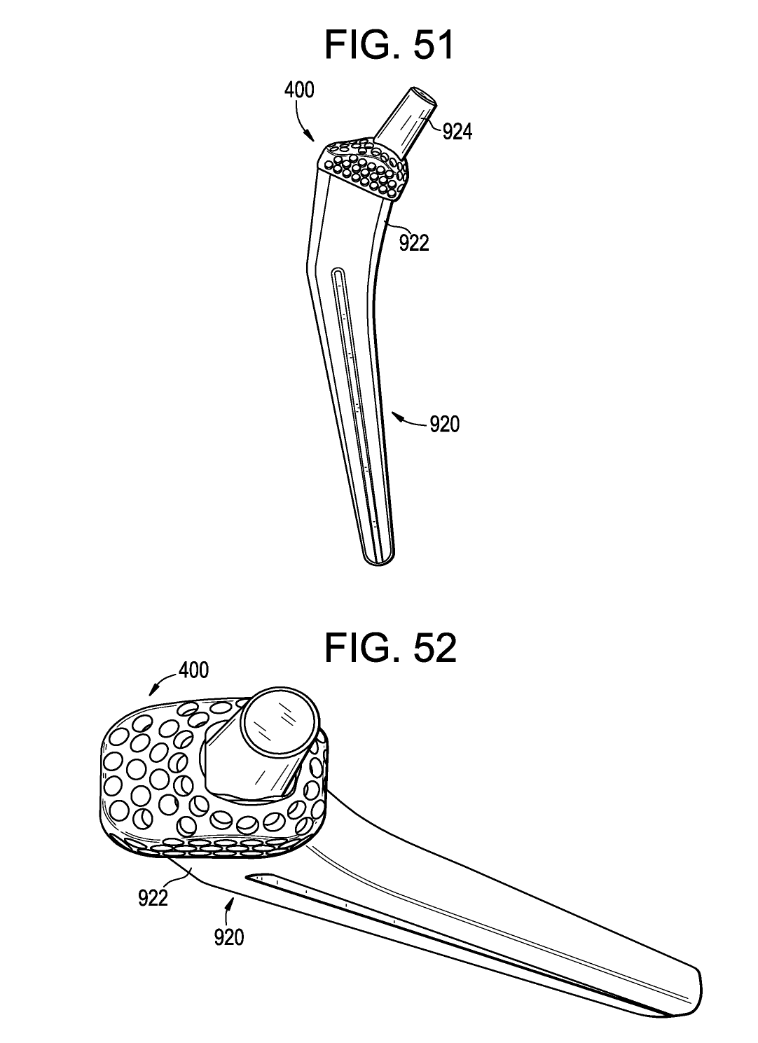

Illustrative, non-limiting examples of the drug delivery devices are described with reference to FIGS. 1-58. In these figures, the drug formulation is not shown in the reservoirs in order to more clearly illustrate the device body and reservoirs.

Device for Knee Prosthetic

FIGS. 1-21 show one embodiment of a drug delivery device 100 adapted for use with a standard knee replacement prosthetic device. The device 100 is designed to be affixed to portion of the knee prosthesis and/or an adjacent surgically prepared bone section, such that the adjacent ligaments and tendons are accommodated. For example, the knee prosthesis may be implanted on a surgically prepared area between the tibia and the femur, and the drug delivery device may be implanted on a tibial portion of the knee prosthesis. Once implanted, the device 100 permits local drug delivery to the implantation site of the knee prosthesis. For example, the drug delivery device 100 may release an antibiotic over the course of several weeks or months to reduce the likelihood of infection associated with the knee prosthesis implantation. The device 100 may be secured with reference to the knee prosthesis to prevent relative movement between the device 100 and the knee prosthesis.

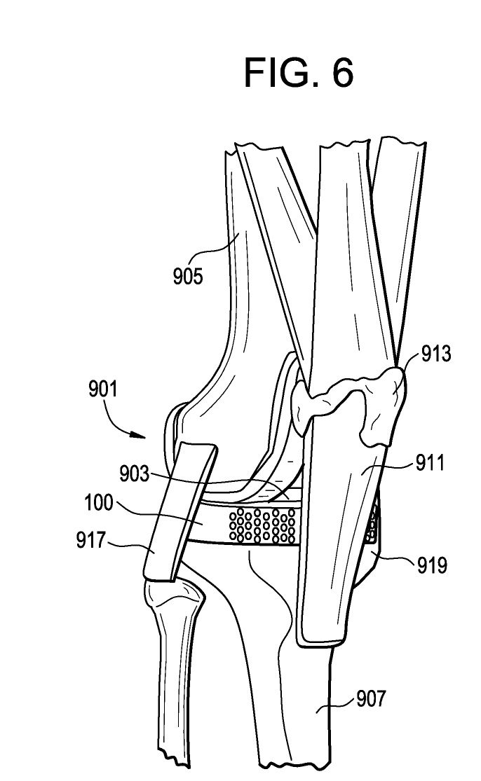

As shown in FIGS. 1-5, which are perspective, top, front, back, and left side views of the device 100, respectively, the device includes a generally C-shaped body 102 having a front portion 104, a back portion 106, a left portion 108, a right portion 110, a top side 112, and a bottom side 114. The directional designations correspond to the orientation of the device once implanted about the knee prosthesis. As shown in FIGS. 6-9, the device 100 is adapted to be implanted about the tibial portion 903 of the knee prosthesis 901, with the top side 112 of the device 100 positioned adjacent to the femur 905, the bottom side 114 positioned adjacent to the tibia 907, the front portion 104 positioned about the articular surface 909 adjacent to the patellar tendon 911 and the patella 913, the back portion 106 positioned adjacent to the posterior cruciate ligament (PCL) 915, the left portion 108 positioned adjacent to the lateral collateral ligament (LCL) 917, and the right portion 110 positioned adjacent to the medial collateral ligament (MCL) 919.

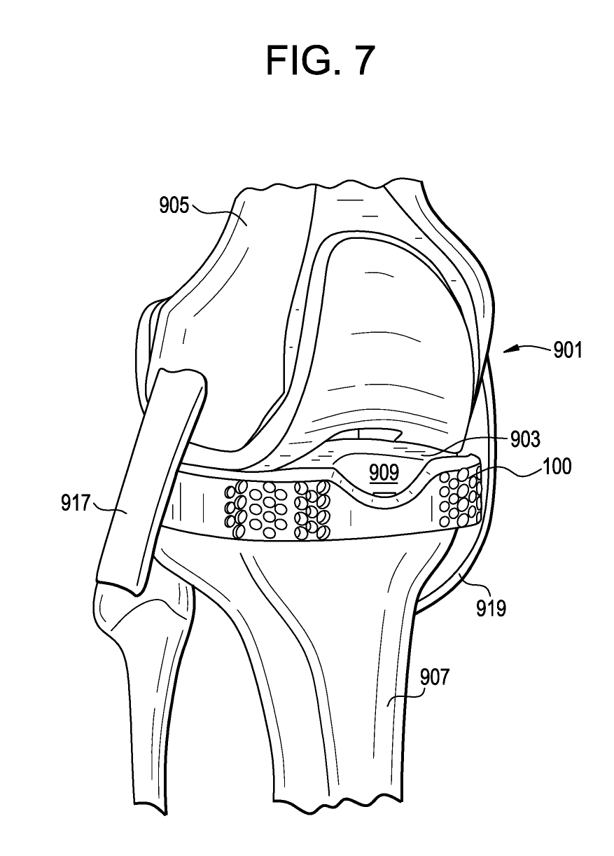

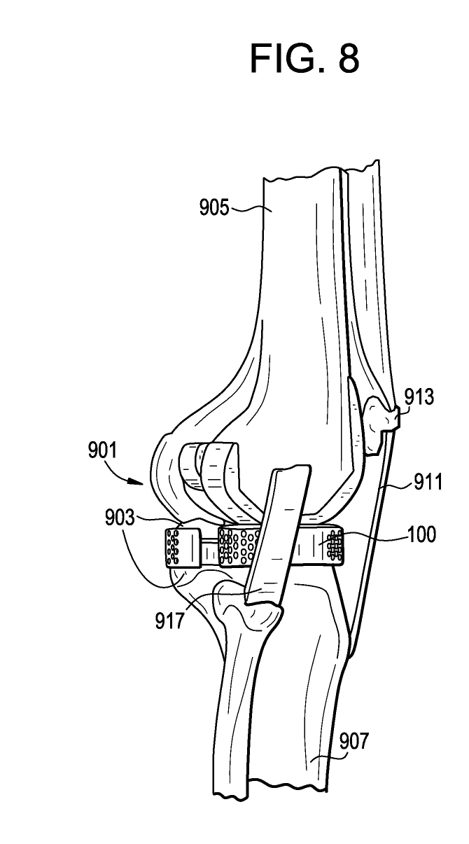

More specifically, FIG. 6 is a front perspective view of the implanted device 100, illustrating the front portion 104 of the device 100 positioned behind the patellar tendon 911 and the left portion 108 of the device 100 positioned behind the LCL 917. FIG. 7 is a front perspective view of the implanted device 100, with the patella and the patellar tendon removed, illustrating the front portion 104 of the device 100 positioned adjacent to the articular surface 909. FIG. 8 is a left rear perspective view of the implanted device 100, illustrating the left portion 108 of the device 100 positioned behind the LCL 917. Although the PCL is not shown, when the device 100 is so implanted the back portion 106 of the device 100 may be out of contact with the PCL. FIG. 9 is a right side view of the implanted device 100 illustrating the right portion 110 of the device 100 positioned beneath the MCL 919.

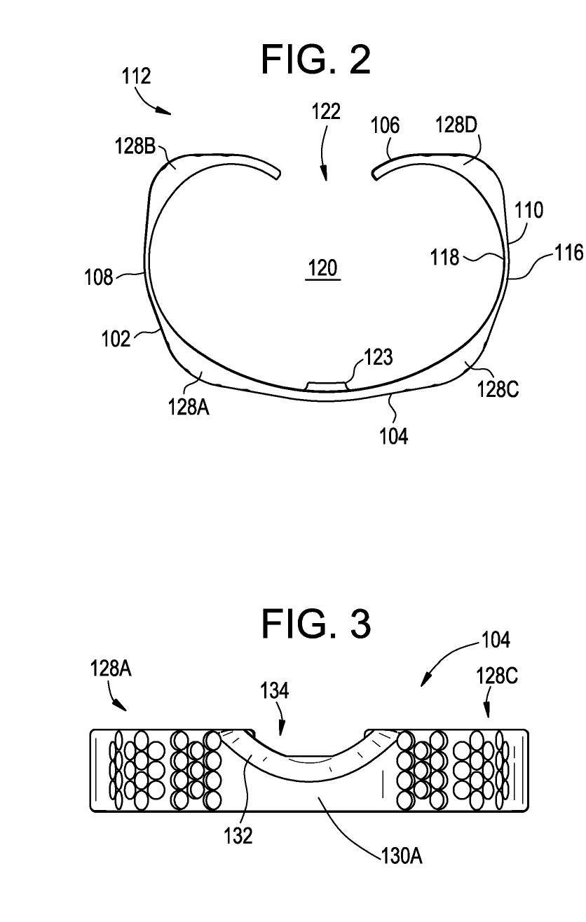

With reference back to FIGS. 1 and 2, The body 102 includes an outer surface 116 and an inner surface 118. The inner surface 118 defines an aperture 120 that is sized and shaped for receiving the tibial portion 903 of the knee prosthesis 901. The aperture 120 extends through the body 102 along a length of the inner surface 118 to permit accommodating the tibial portion 903 of the knee prosthesis 901 within the aperture 120. The body 102 surrounds and substantially encloses the aperture 120 except at a back gap area 122 positioned on the back portion 106, which permits associating the device 100 with the tibial portion 903. More specifically, the body 102 may be adapted to slide over the tibial tray and the articular surface 909.

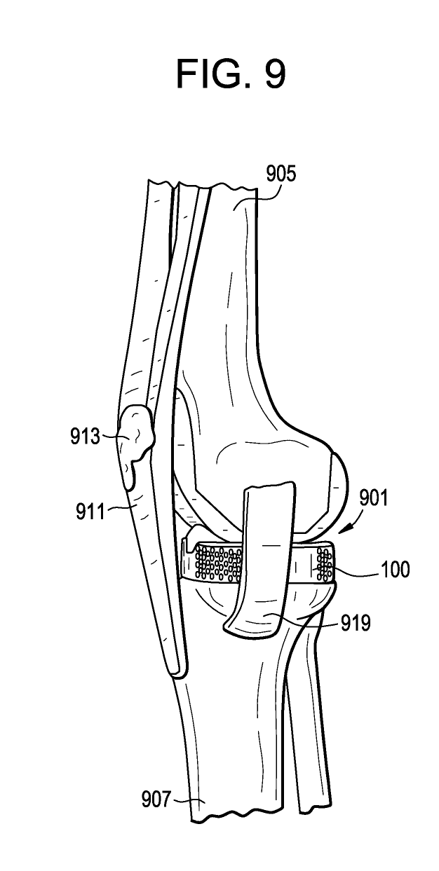

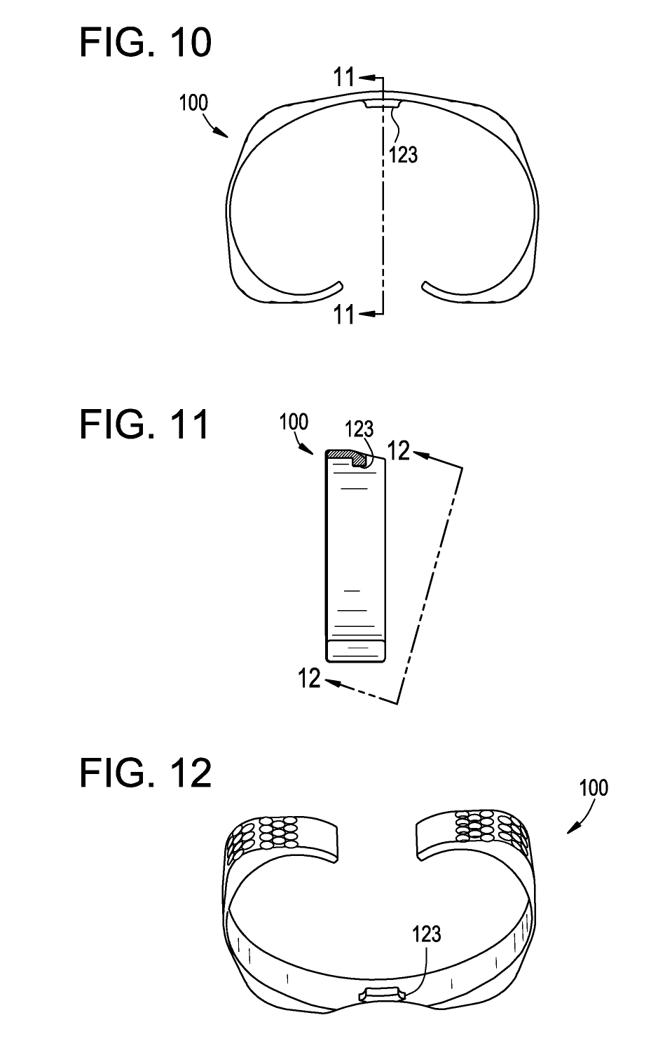

The inner surface 118 may include a locking pin 123 adapted to lock the body 102 about the tibial portion 903. The locking pin 123 may be sized and shaped to mate with a groove in the tibial portion 903. For example, the locking pin 123 may be positioned on the inner surface 118 of the front portion 104 of the device 100, in which case the groove (not shown) may be formed on the articular surface 909 of the knee prosthesis 901. An embodiment of the locking pin 123 can be seen in FIGS. 10-12, where FIG. 11 is a cross-sectional view of the device 100 taken along line C-C of FIG. 10, and FIG. 12 is a rear perspective view of the device 100 taken along line D-D in FIG. 10. Additionally, the body 102 also may be epoxied to the articular surface in addition or as an alternative to the locking pin 123, and/or the body 102 may be secured to the knee prosthesis using any of the fixation means described above.

The outer surface 116 has a number of openings 124 formed through it. The openings 124 may be in communication with a number of discrete reservoirs 126 formed in the device body 102. The reservoirs 126 may store one or more drug formulations that can be released through the openings 124 into the implantation site. In one embodiment, each reservoir 126 may be associated with one opening 124 in the outer surface 116. The orientation of the openings 124 along the outer surface 116 may permit releasing the drug formulation in radially outward directions. The reservoirs 126 are also referred to herein as “wells” or “pockets”.

In embodiments, the reservoirs 126 may be located in groupings or arrays. In some embodiments, the reservoirs 126 may be located in lobe regions 128 positioned about the device body 102. For example, in the illustrated embodiment, the reservoirs 126 are located within four lobe regions 128, including a front left lobe region 128A, a rear left lobe region 128B, a front right lobe region 128C, and a rear right lobe region 128D. The front left lobe region 128A is positioned at the intersection of the front portion 104 and the left portion 108 of the device body 102. The rear left lobe region 128B is positioned at the intersection of the left portion 108 and the back portion 106 of the device body 102. The front right lobe region 128C is positioned at the intersection of the front portion 104 and the right portion 110 of the device body 102. The rear right lobe region 128D is positioned at the intersection of the right portion 110 and the back portion 106 of the device body 102.

In each of these lobe regions 128, the body 102 may have a number of reservoirs 126, and the outer surface 116 of the body 102 may have a number of openings 124 that permit releasing the drug formulation from the corresponding reservoirs 126 into the implantation site. The size, number, and position of reservoirs 126 may be selected to permit storing and releasing a predefined amount of the drug formulation from a given lobe region 128 or from the body 102 has a whole. The size, number, and position of reservoirs 126 may also be selected based on the dimensions of the lobe region 128. More specifically, about the lobe region 128 the body 102 may have a thickness in the radial direction that is selected to accommodate the reservoirs 126 within an interior of the body 102. (The thickness also may be selected based on the available space about the knee prosthesis, although a corresponding region about the human knee is generally sufficiently sized to not present a design limitation.)

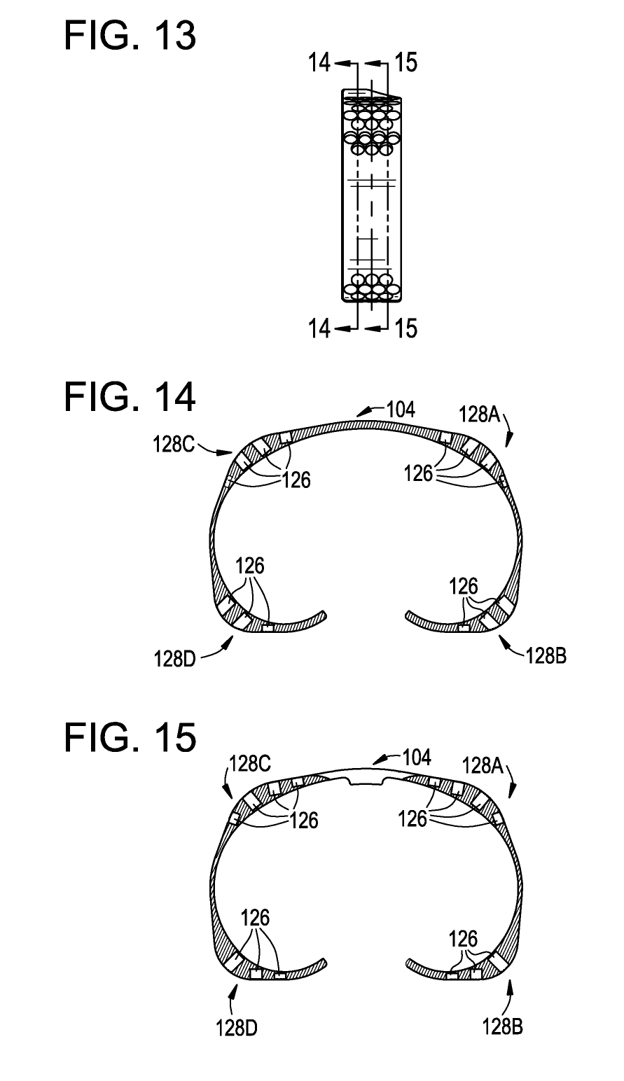

For example, FIG. 14 is a cross-sectional view of the device 100, taken along the line A-A in FIG. 13, and FIG. 15 is a cross-sectional view of the device 100, taken along the line B-B in FIG. 13. As shown, each lobe region 128 may have a thickness that varies or tapers about the periphery of the body 102 from a relatively wider central point to relatively narrower outer points. Due to the difference in thickness across the lobe region 128, the interior of the device body 102 may have a varying amount of space for accommodating the reservoirs 126, and therefore the reservoirs 126 may have varying cross-sectional shapes and sizes, with reservoirs 126 that are centrally positioned being relatively larger than reservoirs 126 positioned about outer edges of the lobe region 128. For example, the device 100 may include reservoirs 126 sized to store a total volume of about 2 mL of the drug formulation, and the openings 124 have diameters of about 3 mm for releasing the drug formulation from the reservoirs 126. In other embodiments, the lobe regions 128 may be coated in a drug-eluting coating, in addition or as an alternative to the reservoirs 126.

In one embodiment, the body 102 may also include a number of low influence regions 130. The low influence regions 130 may be configured to minimize or avoid irritating a tendon or a ligament. For example, the outer surface 116 of the body 102 may be relatively smooth in the low influence regions 130. The thickness of the body 102 in the radial direction may be relatively narrow in the low influence regions 130. The body 102 may also be substantially free from reservoirs 126 in the low influence regions 130, and the outer surface 116 may not include openings 124.

The low influence regions 130 may be positioned about the body 102 spaced apart from the lobe regions 128. Specifically, the low influence regions 130 may be positioned such that when the device 100 is implanted about the knee prosthesis, the low influence regions 130 become positioned adjacent to the tendons and ligaments and the lobe regions 128 become positioned spaced apart from the tendons or ligaments, so that irritation of the tendons and ligaments is reduced or eliminated.

In the illustrated embodiment, the low influence regions 130 include a patellar tendon low influence region 130A, a LCL low influence region 130B, a MCL low influence region 130C, and a PCL low influence region 130D. The patellar tendon low influence region 130A is positioned on the front portion 104 of the body 102 between the front left lobe region 128A and the front right lobe region 128C. The LCL low influence region 130B is positioned on the left portion 108 of the device body 102 between the front left lobe region 128A and the rear left lobe region 128B. The MCL low influence region 130C is positioned on a right portion 110 of the body 102 between the front right lobe region 128C and the rear right lobe region 128D. The PCL low influence region 130D is positioned on the back portion 106 of the body 102 between the rear left lobe region 128B and the rear right lobe region 128D. In each of these low influence regions 130, the body 102 is relatively narrower in the radial direction, the outer surface 116 is relatively smooth, and the body 102 is substantially free from reservoirs 126. Additionally, the patellar tendon low influence region 130A may include a tapered area 132 over which the thickness of the body 102 further reduces to become a front cut-away area 134. The tapered area 132 and front cut-away area 134 may further accommodate the patellar tendon 911 and the patella 913. Similarly, the back gap area 122 further provides a relief area for accommodating the PCL. When the device 100 is implanted, the patellar tendon low influence region 130A becomes positioned adjacent to the patellar tendon 911, the LCL low influence region 130B becomes positioned adjacent to the LCL 917, the MCL low influence region 130C becomes positioned adjacent to the MCL 919, and the PCL low influence region 130D becomes positioned adjacent to the PCL 915.

In alternative embodiments, the low influence regions 130 may include an array of sufficiently small reservoirs and openings, such as micro-reservoirs and associated micro-openings. The micro-reservoirs may contain one or more drug formulations or other substances, which may be released through the micro-openings without substantially irritating the adjacent tendons or soft tissue.

The body 102 may be of a unitary construction. The body 102 may formed of a biocompatible material, such as a metal, a polymer, a ceramic, or a combination thereof as described herein. The biocompatible material may or may not be biodegradable or bioerodible. For example, the body 102 may be formed from a biodegradable or bioerodible polymer.

In one embodiment, the body 102 may be formed of, for example, a biocompatible metal, a non-biodegradable polymer of sufficiently rigidity, a non-biodegradable ceramic, a degradable polymer, or a combination thereof. In some embodiments, the body 102 may be formed from a core covered in a drug-eluting coating. The coating may be a blend of a polymer and a drug, such that the drug is released from the coating in vivo. In embodiments in which the body 102 has a core, the coating may cover the lobe regions 128 and the reservoirs 126 may be omitted. Alternatively, the body 102 may have the reservoirs 126 and the drug-eluting coating.

The body 102 may also have a variety of different sizes to accommodate conventional knee prosthesis of varying sizes. For example, FIGS. 16-21 are perspective, front, rear, top, bottom and left side views, respectively, of an embodiment of the drug delivery device shown in FIG. 1, illustrating relative dimensions of the drug delivery device. Actual dimensions are provided, by way of example, on corresponding figures shown in the U.S. Provisional Application No. 60/975,504, which was filed on Sep. 26, 2007 and is incorporated by reference above. To accommodate knee prosthesis of other sizes, the body 102 may be made relatively smaller or larger, such as by varying one or more dimensions of the device 100. It should be noted that devices 100 of different sizes may have different locations, sizes, and numbers of reservoirs to accommodate the desired volume of drug formulation while maintaining the mechanical integrity of the device.

The device 100 may be incorporated into a knee replacement procedure, such as a total knee replacement arthroplasty (TKA) procedure, with relative ease. The device 100 may permit controlled release of any number of drug formulations, including antibiotics, into the implantation site. Further, the device may not impinge against the knee prosthesis or adjacent anatomy upon movement.

Device for Hip Prosthetic

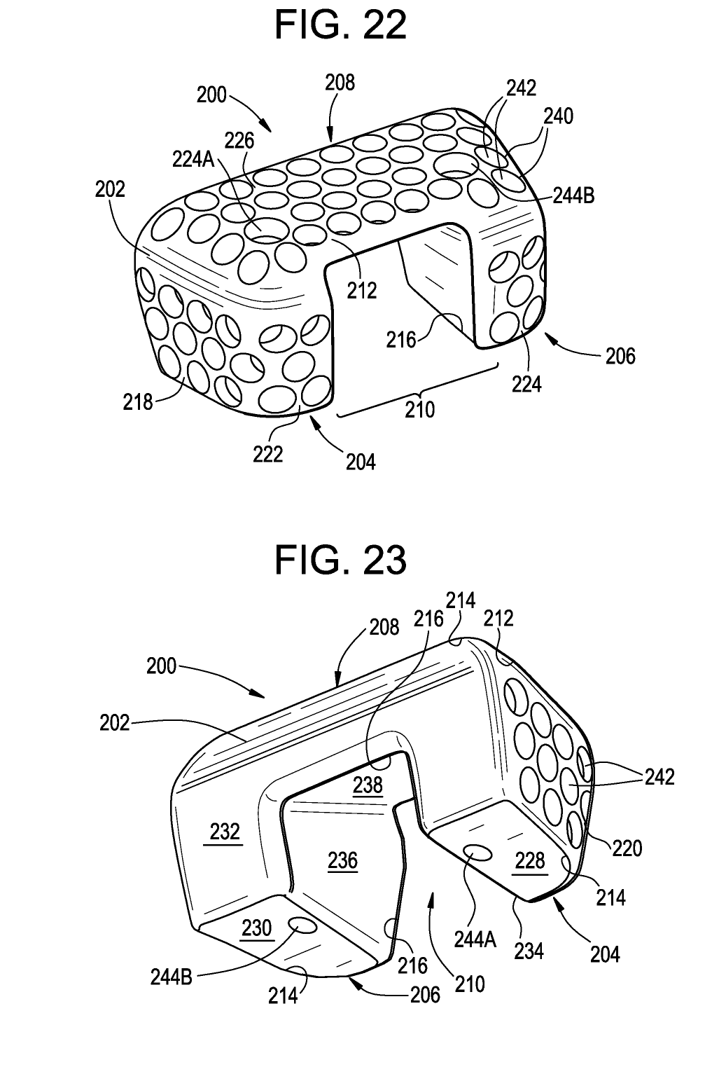

FIGS. 22-30 show embodiments of a drug delivery device 200 for use with, for example, a standard hip replacement prosthesis. In embodiments, the drug delivery device 200 is designed to be positioned adjacent to a hip stem of the hip prosthesis. More specifically, the drug delivery device 200 may be implanted between a surgically prepared portion of the femur and the hip stem of the implanted hip prosthesis. Once implanted, the drug delivery device 200 permits local drug delivery to the implantation site of the hip prosthesis. For example, the drug delivery device 200 may release an antibiotic over the course of several weeks or months, to reduce the likelihood of infection associated with the implantation of the hip prosthesis. The device 200 may be secured with reference to the hip prosthesis to prevent relative movement between the device 100 and the hip prosthesis. The drug delivery device 200 may also be suited for use with other prosthetic implants or parts of the anatomy in general.

As shown in FIGS. 22-23, which are upper and lower perspective views of the drug delivery device 200, the device 200 includes a generally bridge shaped or arcuate body 202. More specifically, the body 202 includes a front pillar 204, a rear pillar 206, and an intermediate cross piece 208 extending between the front pillar 204 and the rear pillar 206 on an upper portion of the device 200. Below the intermediate cross piece 208, an aperture or slot 210 is defined on a lower portion of the device 200.

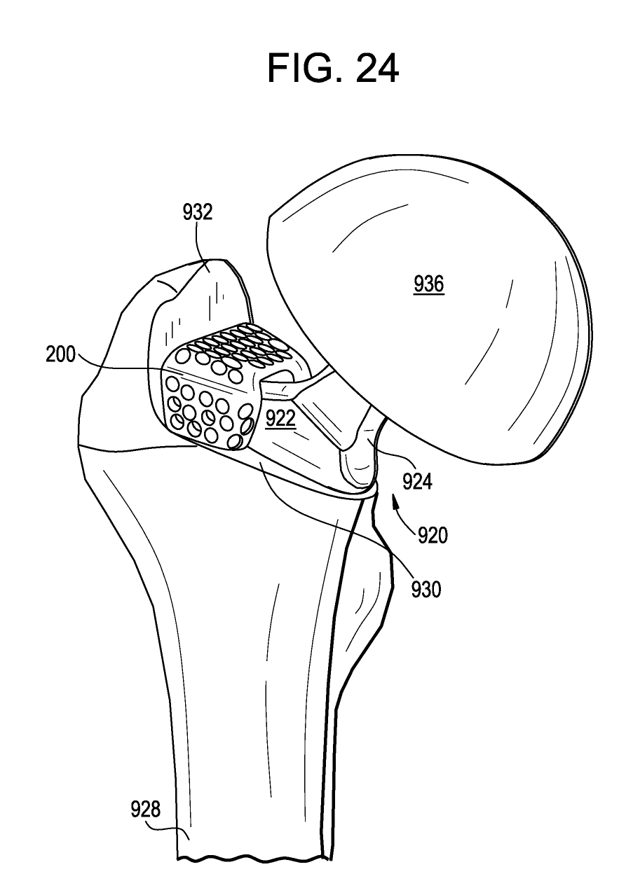

Thus, the device 200 is shaped to be implanted about a hip prosthesis 920, as shown in FIGS. 24-30. As shown, the hip prosthesis 920 generally includes a hip stem 922, a neck 924, and a head or ball 926. The head 926 may be associated with an acetabular cup 936, which is designed to be positioned in a socket of the pelvis. The hip stem 922 may be an unmodified, tapered hip stem. The hip prosthesis 920 is normally implanted on a section of the femur 928 that has been surgically prepared, such as by cutting the femur 928 to remove at least a portion of the femoral head, thereby forming a lower cut surface 930 and a lateral cut surface 932. Typically, the cut surfaces 930, 932 are relatively planar and form an included angle of greater than 90°, such as an angle of about 146°, although other angles are possible. The hip prosthesis 920 is then implanted in the femur 928 with the hip stem 922 extending through the lower cut surface 930, the neck 924 protruding from the hip stem 922 toward the pelvis, and the head 926 positioned on an end of the neck 924 for association with the acetabular cup 936 and the pelvis.

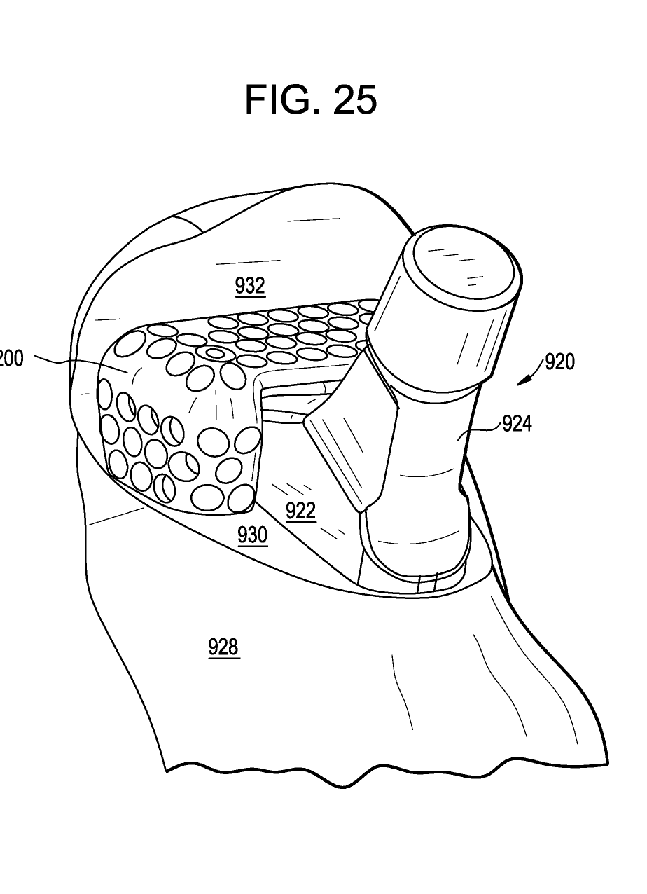

After the hip prosthesis 920 is so implanted, the drug delivery device 200 may be implanted about the hip prosthesis 920. FIG. 24 is a perspective view of the drug delivery device 200 positioned about a portion of an implanted hip prosthesis 920, such as on the surgically prepared portion of the femur 928 in a space formerly occupied by the femoral head. The front pillar 204 may be positioned in front of the hip stem 922, the rear pillar 206 may be positioned behind the hip stem 922, and the intermediate cross piece 208 may extend above the hip stem 922 adjacent to the neck 924. The aperture 210 may be accommodate the hip stem 922 as shown in FIG. 25, which is another perspective view of the drug delivery device 200 positioned about a portion of an implanted hip prosthesis 920, illustrating the hip stem 922 with the head 926 removed. More specifically, the aperture 210 may receive the implant in confronting relation around three sides of the hip stem 922.

It should be noted that directional designations, such as front, back, left, right, forward, rearward, upper, lower, and lateral, among others, are provided for explanatory purposes. These directions correspond to the orientation of the recited component with reference to a left leg positioned with a left foot on the floor. Of course, the relative orientation of the hip prosthesis 920 and the drug delivery device 200 will vary with movement of the leg. Therefore, these directional designations are not intended to limit the disclosure. Further, the device 200 is illustrated and described with reference to the left leg, although the device 200 may be suited for use with either leg merely by rotating the device 180°. In such cases, the directional designations referenced above are 180° out of phase. The device 200 may also be suited for implantation about other portions of the anatomy or prosthetic implants.

With reference back to FIGS. 22-23, the device body 200 is generally bounded by an outer or exposed surface 212, inner or occluded surfaces 214, and a partially occluded surface 216. The exposed surface 212 is outwardly directed and is adapted to permit releasing a drug formulation into a targeted area, such as the implantation site of a hip prosthesis 920. The occluded surfaces 214 are inwardly directed and are adapted to be positioned in confronting relation to a surgically prepared bone section, such as one or more of the cut surfaces 930, 932 of the femur 928. The partially occluded surface 216 is adapted to be positioned about or in close proximity to a portion of the prosthetic implant 920, such as the hip stem 922 of the hip prosthesis 920.

The exposed surface 212 is best seen in FIG. 22, and generally includes a front face 218 of the front pillar 204, a rear face 220 of the rear pillar 206, right side faces 222, 224 of the front and rear pillars 204, 206, and an upper face 226 extending along the front pillar 204, the intermediate cross piece 208, and the rear pillar 206. The body 202 is generally curved at intersections of these faces such that the exposed surface 212 is relatively free of sharp edges. The exposed surface 212 is “exposed” in the sense that, once the device 200 is implanted in the body, the exposed surface 212 is generally exposed to the implantation environment, such that the drug formulation released through the exposed surface 212 may have the intended effect at the implantation site.

The occluded surfaces 214 are best seen in FIG. 25. The occluded surfaces 214 generally includes lower faces 228, 230 of the front and rear pillars 204, 206, and a left side face 232 extending along the front pillar 204, the intermediate cross piece 208, and the rear pillar 206. The occluded surfaces 214 are “occluded” in the sense that, once the device 200 is implanted in the body, the occluded surfaces 214 are positioned in confronting relation to the surgically prepared bone sections, such as the lower and lateral cut surfaces 930, 932 of the femur 928.

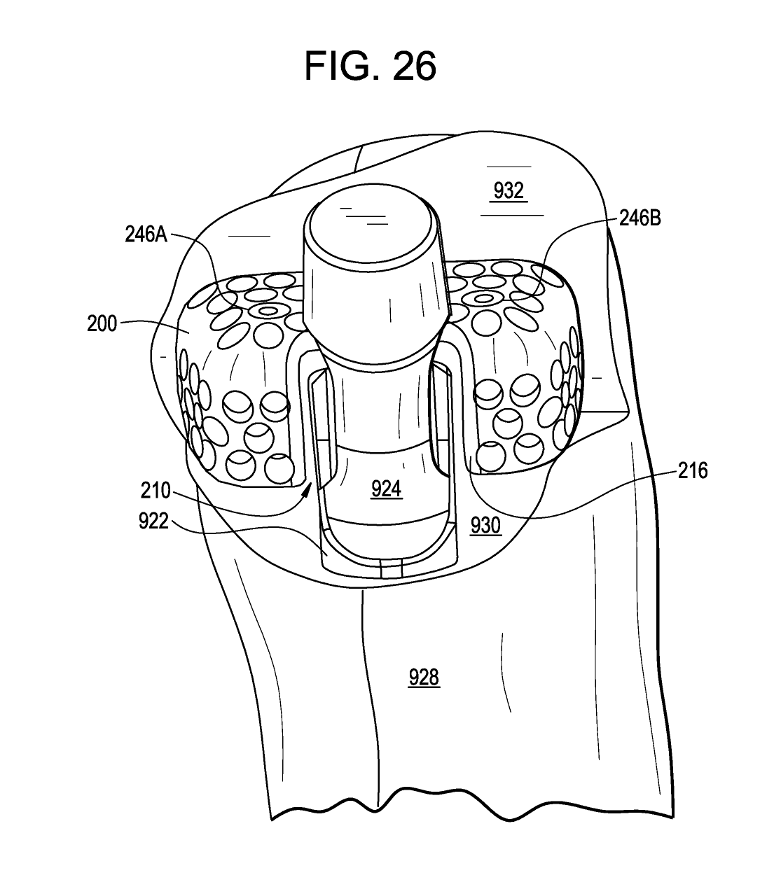

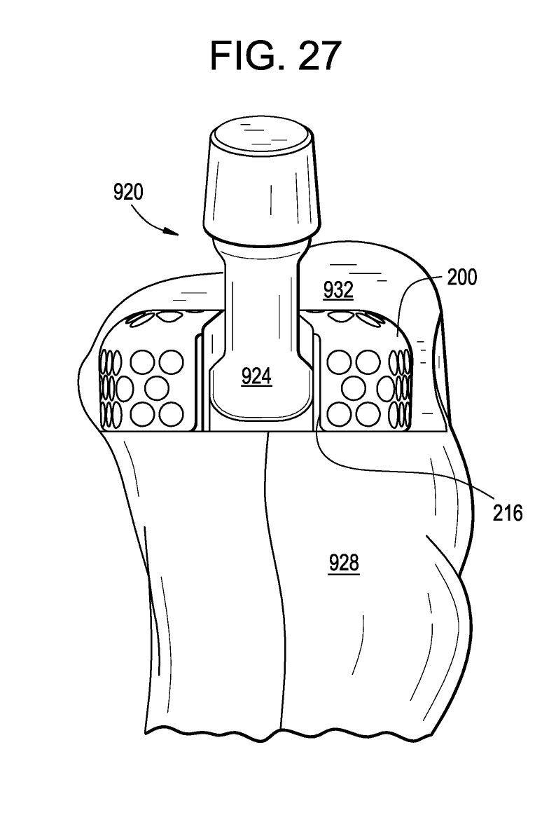

The partially occluded surface 216 includes a rear face 234 of the front pillar 204, a front face 236 of the rear pillar 206, and a lower face 238 of the intermediate cross piece 208. The partially occluded surface 216 is “partially occluded” in the sense that, once the device 200 is implanted in the body, the partially occluded surface 216 is adjacent to and in close proximity to a portion of the prosthetic implant 920, such as the hip stem 922, but is at least slightly spaced apart from contact with the portion of the prosthetic implant 920. Such positioning of the partially occluded surface 216 is best seen in FIGS. 26-27, which are side views of the device 200 implanted adjacent to the hip stem 922 with the head 926 removed for illustrative purposes.

The device 200 may be adapted to release a drug formulation into the implantation site once implanted, such as through the exposed surface 212. With reference back to FIG. 22, the exposed surface 212 may have a number of openings 240 formed through it. The openings 240 may be in communication with a number of discrete reservoirs 242 formed in the device body 202. The reservoirs 242 may store one or more drug formulations that can be released through the openings 240 into the implantation site. The drug formulation is not shown for clarity. In one embodiment, each reservoir 242 may be associated with one opening 240 in the exposed surface 212. The orientation of the openings 240 along the exposed surface 212 may permit releasing the drug formulation in radially outward directions. Thus, when the device 200 is implanted, the drug formulation may be released from the reservoirs 242 through the exposed surface 212 via the openings 240 and into tissues and fluids at the implantation site.

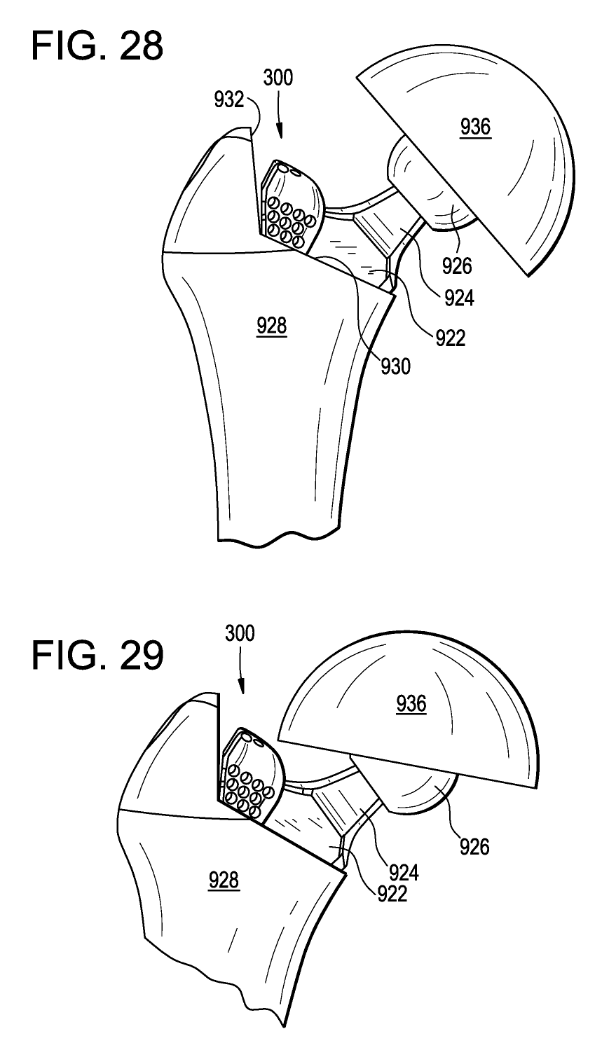

The device 200 may be sized for use with commercially available hip prostheses of various sizes. More specifically, the pillars 204, 206 may be sized such that once the device 200 is implanted, the upper face 226 is even with or lower than the remaining portion of the femur 928. Such sizing reduces the likelihood of the device 200 interfering with remaining portions of the anatomy, such as adjacent muscles, tendons, and ligaments. The aperture 210 may also be sized to accommodate hip stems 922 of various dimensions and configurations, with the partially occluded surface 916 being spaced apart from the hip stem 922 to prevent mechanical interaction between the device 200 and the hip stem 922. The upper face 226 of the body 200 may slope slightly away from the left side face 232, so that the acetabular cup 936 may not impinge against the drug delivery device 200 as the leg is rotated. For example, the average abduction angle permitted by an implanted hip prosthesis 920 is about 25 degrees, while the device 200 may permit rotation of as much as 45 degrees without impingement occurring. Such a configuration is shown in FIGS. 28-29, which are front views of another embodiment of a drug delivery device 300 (described in further detail below) illustrating the device 300 implanted adjacent to a portion of an implanted hip prosthesis 920, with the acetabular cup 936 in a non-rotated position and rotated position, respectively, the acetabular cup 936 not impinging against the drug delivery device 300 when in the rotated position despite a large degree of rotation about the head 926.

Because the cut surfaces 930, 932 are generally cut in a roughly planar configuration during the surgical preparation, the occluded surfaces 214, including the lower faces 228, 230 of the pillars 204, 206 and the left side surface 232, may be generally planar. Each lower face 228, 230 may form an angle with the left side face 232. The angle may substantially correspond to the included angle between the lower cut surface 930 and the lateral cut surface 932, so that the occluded surfaces can be placed in confronting relation to the surgically prepared bone section. For example, the angle may be about greater than 90 degrees, such as at about 146 degrees in some embodiments, although other configurations are possible.

The size and shape of the device 200 may permit housing the reservoirs 242, and therefore the drug formulation, within the body 202. The reservoirs 242 may have varying cross-sectional shapes and sizes depending on the position on the body 202, as varying amount of space may be available for accommodating the reservoirs 242 within the body 202. For example, each reservoir 242 may have a diameter of about 3 mm and a depth varying from about 1.35 mm to about 9 mm, depending on the location on the body 202. In total, the reservoirs 242 may be sized to store a total volume of about 2 mL of the drug formulation, which may be an antibiotic. The reservoirs 242 may be in communication with the openings 240 located about the exposed surface 212, such as on the front face 218 of the front pillar 204, and the rear face 220 of the rear pillar 206, the right side faces 222, 224 of the pillars, and the upper face 226. In the embodiment shown, reservoirs 242 are not provided on the occluded surfaces 214 or the partially occluded surface 216. For example, the occluded surfaces 214 may be relatively free from reservoirs 242 to avoid releasing drug formulation onto adjacent portions of the of the femur 928, which may be irritated. The partially occluded surface 216 may also be relatively free from reservoirs 242, so that the drug formulation is generally released into the implantation site instead of onto the hip stem 922. However, the occluded and/or partially occluded surfaces 214, 216 may have reservoirs, such as microreservoirs, in other embodiments.

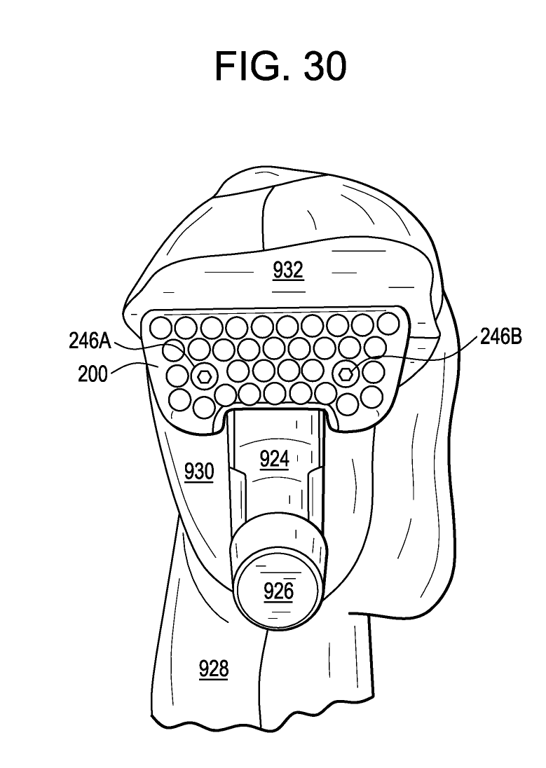

The device 200 may be secured to the surgically prepared bone section and/or the implanted prosthesis in a variety of manners. In the embodiment illustrated in FIGS. 22-30, for example, the device 200 is adapted to be attached to the surgically prepared portion of the femur with a number of screws. The body 202 may include a number of screw holes 244 that extend substantially vertically through the device body 202, and a corresponding number of screws 246, such as conventional bone screws, may be positioned in the screw holes 244. As shown in FIGS. 22-23, for example, a front screw hole 244A may extend through the front pillar 204 from the upper face 226 through the lower face 228, and a rear screw hole 244B may extend through the rear pillar 206 from the upper face 226 through the lower face 230. As shown in FIGS. 26 and 30, a front screw 246A may be positioned in the front screw hole 244A to secure the front pillar 204 to the lower cut surface 930 and a rear screw 246B may be positioned in the rear screw hole 244B to secure the rear pillar 206 against the lower cut surface 930. However, in other embodiments, any number or positioning of screw holes 244 and corresponding screws 246 may be used.

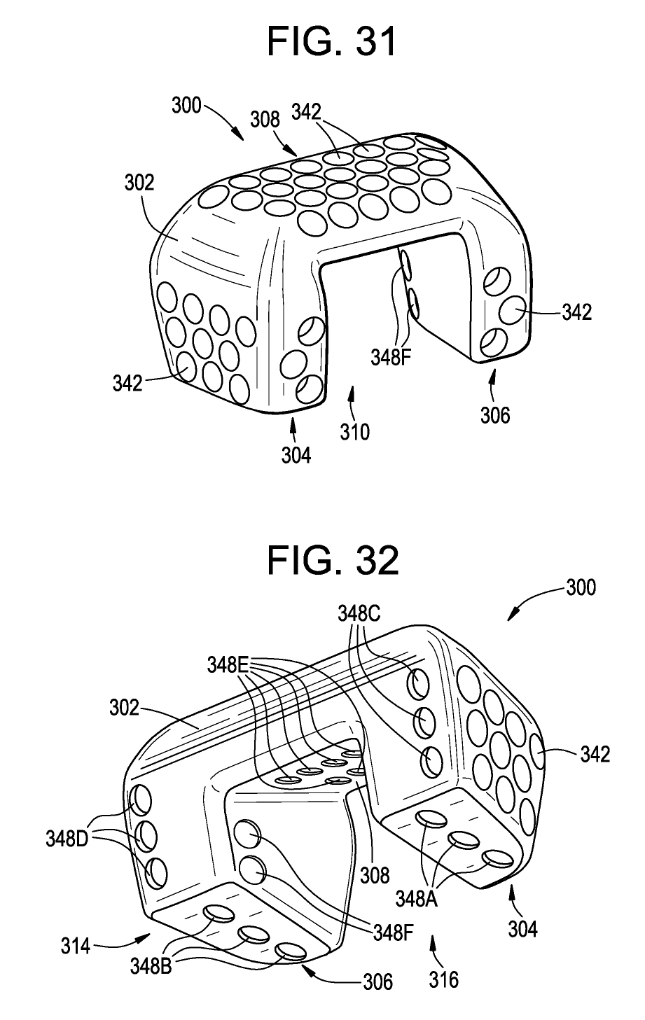

Further, the device may also be secured in other manners. For example, FIGS. 31-38 illustrate another embodiment of a drug delivery device 300. Although the device 300 is substantially similar to the device 200, the device 300 is adapted to be secured with bone cement. More specifically, the body 302 may include a number of cement holes 348 that extend into the device body 302, and a bone cement, such as conventional bone cement, may be positioned in the cement holes 348. The cement holes 348 may be formed through the pillars 304, 306 and the intermediate cross piece 308. The cement holes 348 may be exposed on the occluded surfaces 314 for securing the device 300 to the cut surfaces 930, 932. The cement holes 348 may also be exposed on the partially occluded surface 316 for securing the device 300 to a portion of the implant, such as the hip stem 922 of the hip prosthesis 920. As shown in FIGS. 31-32, for example, a set of front vertical cement holes 348A may extend into the front pillar 304 from the lower face, and a set of rear vertical cement holes 348B may extend into the rear pillar 306 from the lower face. Similarly, a set front and rear lateral cement holes 348C, 348D may extend into the front and rear pillars 304, 306, respectively, from the corresponding the left side face. A set of intermediate vertical cement holes 348E may extend into the intermediate cross piece 308 from the lower face, and a set of intermediate lateral cement holes 348F may extend through the front and rear pillars 304, 306 from the inner faces. Bone cement may be positioned in the front and rear vertical cement holes 348A, 348B to secure the front and rear pillars 304, 306 to the lower cut surface 930, in the front and rear lateral cement holes 348C, 348D to secure the front and rear pillars 304, 306 to the lateral cut surface 932, in the intermediate vertical cement holes 348E to secure the intermediate cross piece 308 to the hip stem 922, and in the intermediate lateral cement holes 348F to secure the front and rear pillars 304, 306 to the hip stem 922. However, any number or positioning of cement holes 348 may be used in other embodiments. Although the drug delivery device 300 is generally similar to the drug delivery device 200, the size and shape of the device 300 may vary slightly to accommodate the cement holes 348, as shown in the figures.

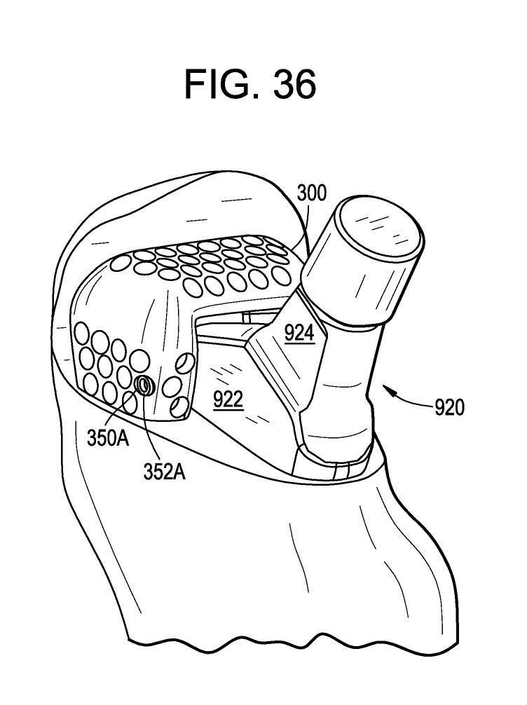

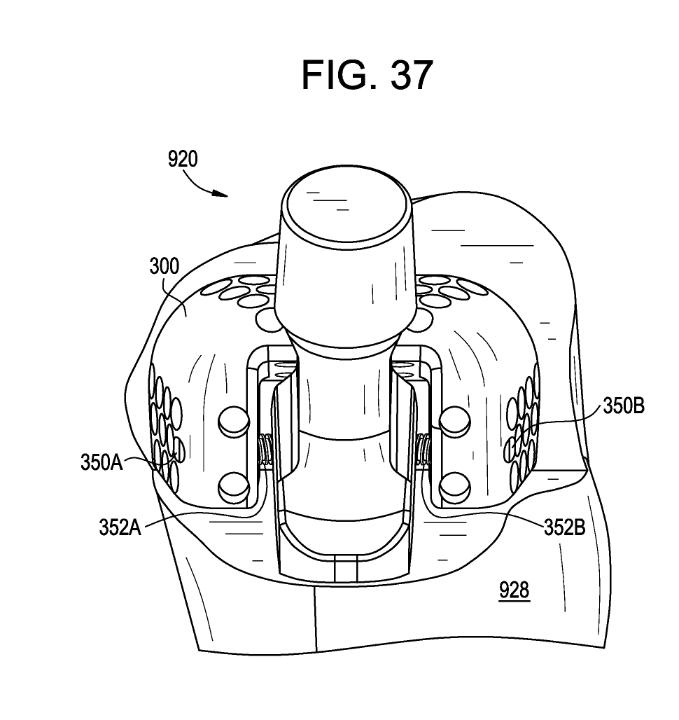

In one embodiment, the device 300 may be further held in place using one or more set screws. FIG. 36-37 are perspective and side views, respectively, of the drug delivery device 300, illustrating set screws maintaining a position of the device 300 against a hip stem 922 of the implanted hip prosthesis 920. As shown, the device 300 may include a forward set screw hole 350A that extends through the front pillar 304 from the front face to the rear face, and a rear set screw hole 350B that extends through the rear pillar 306 from the rear face to the front face. Set screws 352A, 352B may be positioned in the corresponding set screw holes 350A, 350B to maintain a position of the device 300 against a hip stem 922 of the implanted hip prosthesis 920. In such embodiments, the device 300 may be reconfigured to accommodate the set screw holes 350, such as by removing one or more of the reservoirs 342 or cement holes 348, although such reconfiguration may not be necessary. It should be noted that set screws may also be used with the device 200, in which case the device 200 may have corresponding set screw holes. Further, any combination of these or other fixation means may be used to secure the device to the surgically prepared bone section and/or the implant, as described above.

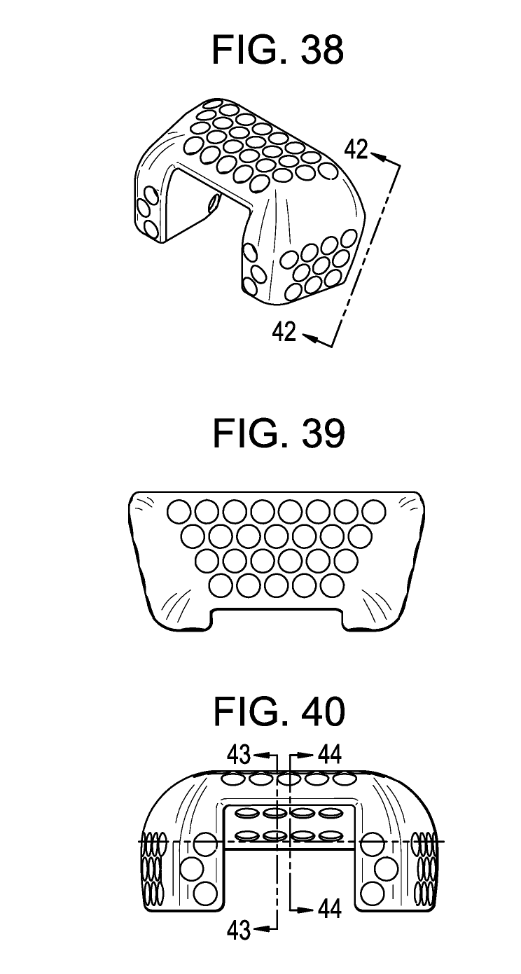

FIGS. 38-48 show various views of the drug delivery device 300, illustrating example relative dimensions of the device. Actual dimensions are provided, by way of example, on corresponding figures shown in the U.S. Provisional Application No. 60/975,504, which was filed on Sep. 26, 2007 and is incorporated by reference above. To accommodate hip prostheses 920 of various sizes, the body 302 may be made relatively smaller or larger, such as by varying one or more dimensions of the device 300, although the illustrated embodiment may be suited for use with a number of hip prostheses 920 of conventional sizes. It should be noted that devices 300 of different sizes may have different locations, sizes, and numbers of reservoirs to accommodate the desired volume of drug formulation while maintaining the mechanical integrity of the device.

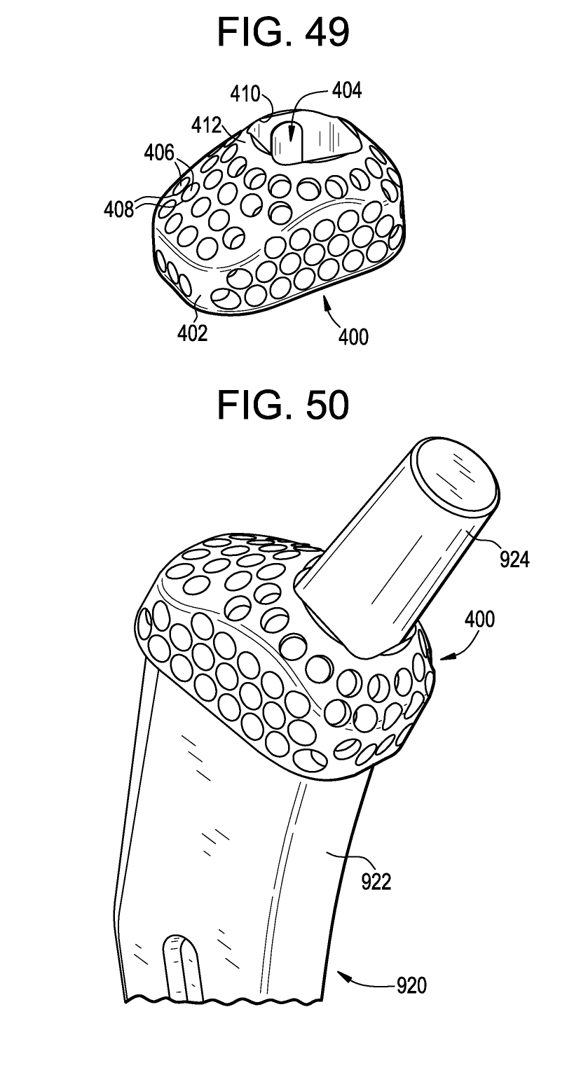

FIGS. 49-55 illustrate another embodiment of a drug delivery device 400 adapted for use with a standard hip replacement prosthesis, such as the prosthesis 920 described above. As shown in FIG. 49, which is a perspective view of the drug delivery device 400, the device 400 may generally include a body 402 formed in the shape of a closed sleeve or cap. As shown in FIGS. 50-55, which illustrate the drug delivery device 400 implanted about a hip prosthesis 920, the cap shape of the body 402 may permit fitting the body 402 over a portion of the implanted prosthesis 920, such as the area formerly occupied by the femoral head. The body 402 may also include a hip stem aperture 404 that may be sized and shaped to receive a portion of the neck 924 of the hip stem 922. The shape of the body 402 may permit installing the device 400 at a height that is even with or lower than existing stem flats on the hip stem 922, for reasons described below. As shown, the hip stem 922 may be an unmodified, tapered hip stem.

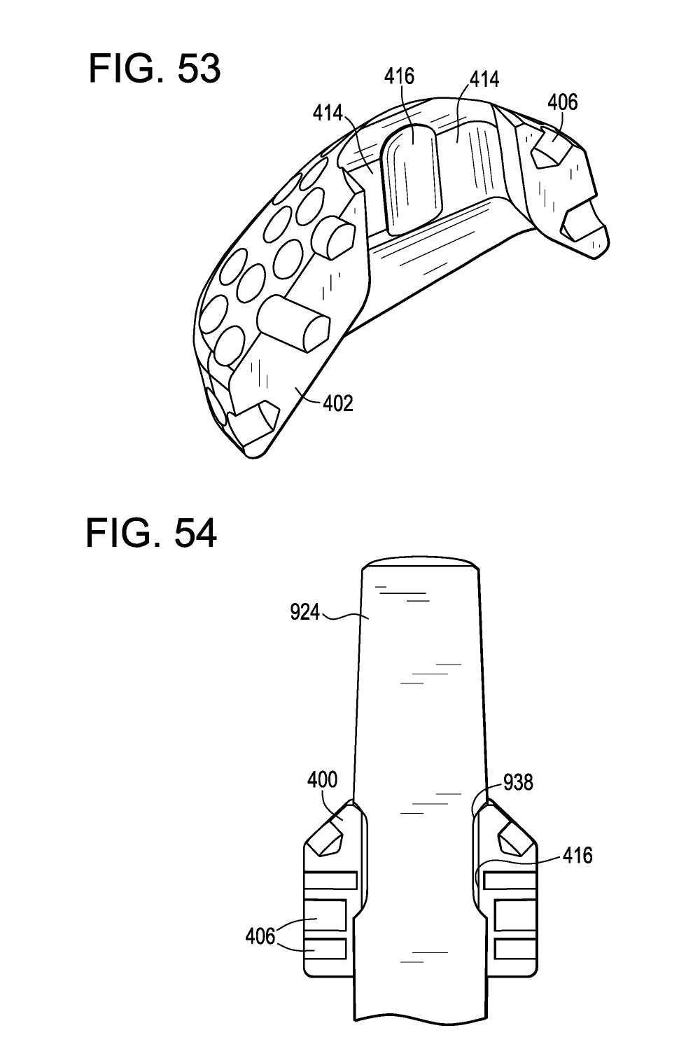

The body 402 may include an inner surface 410 that defines the aperture 404. Thus, the inner surface 410 may be sized and shaped for placement in confronting relation to the neck 924 of the hip stem 922. The body 402 may also include an outer surface 412 that is outwardly directed toward the adjacent anatomy once the device 400 is implanted. A number of reservoirs 406 may be formed in the body 402 for containing a drug formulation. The reservoirs 406 may be in communication with openings 408 formed in the outer surface 412, so that the drug formulation may be released from the reservoirs 406 through the openings 408 and into the implantation site. The reservoirs 406 may have varying cross-sectional shapes and sizes depending on the position on the body 402, as a varying amount of space may be available for accommodating the reservoirs 406 within the body 402. For example, each reservoir 406 may have a diameter of about 3 mm and a depth varying from about 2.8 mm to about 5 mm, depending on the location on the body 402. The reservoirs 406 of varying sizes and shapes can be seen in FIG. 53, which is a perspective cross-sectional view of the device 400. In total, the reservoirs 406 may be sized to store a total volume of about 2 mL of the drug formulation, which may be an antibiotic.

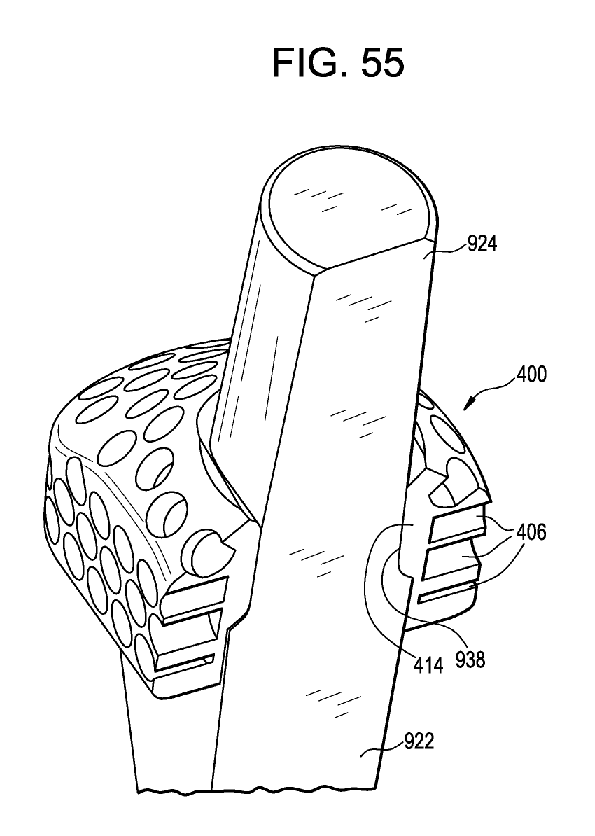

As shown in FIG. 53, the device 400 may include one or more protruding flat areas 414 and one or more relief areas 416. The protruding flat areas 414 and the relief areas 416 may be positioned in the hip stem aperture 404. The protruding flat areas 414 may be adapted to engage stem flats 938 on the neck 924 of the hip stem 922 to secure the device 400 to the hip stem 922, and the relief areas 416 may permit the device 400 to deflect so that the device 400 may be slid onto the neck 924 of the hip stem 922 despite the protruding flat areas 414. For example, FIG. 54 is a front cross-sectional view of the device 400 implanted about the hip stem 922, taken through the relief areas 416 positioned adjacent to the stem flats 938, and FIG. 55 is a perspective cross-sectional view of the device 400 implanted about the hip stem 922, taken through the protruding flat areas 414 positioned adjacent to the stem flats 938.

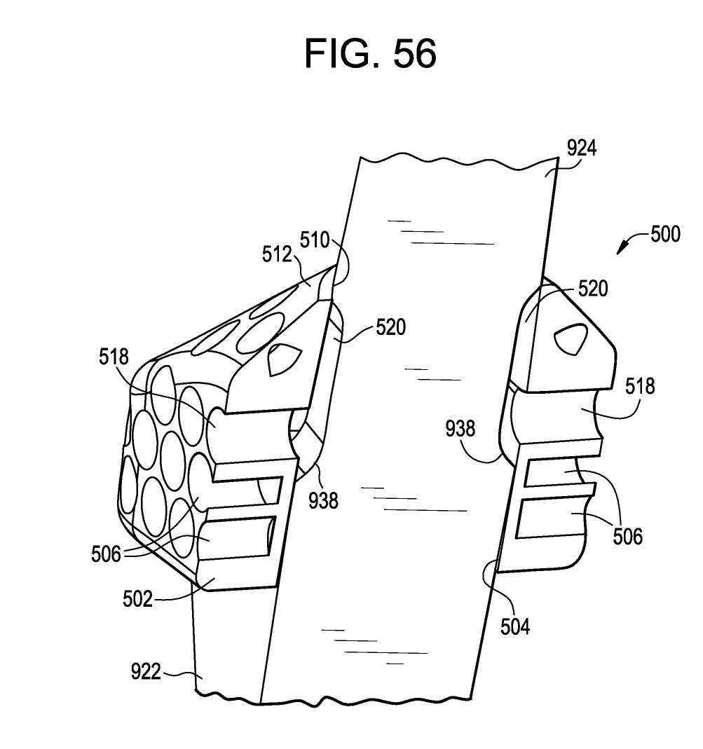

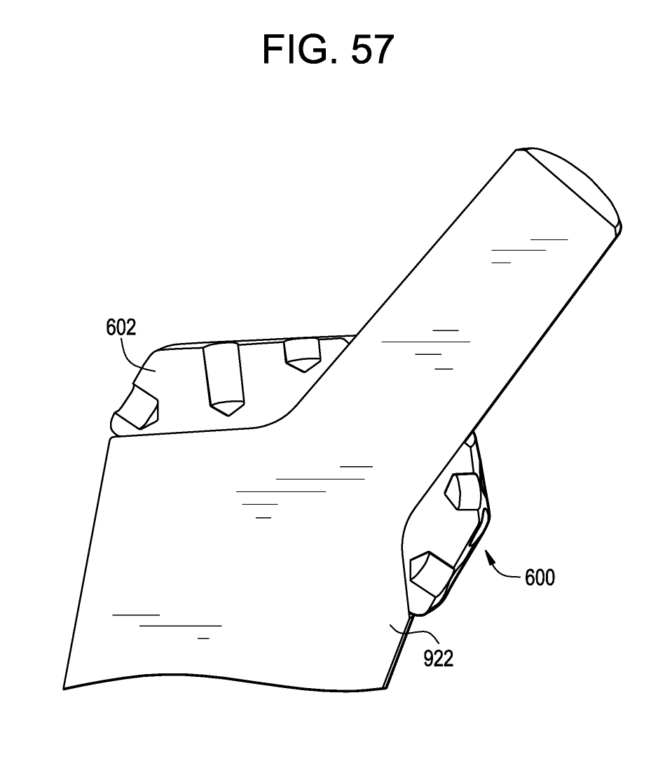

However, the device 400 may be secured to the hip stem 922 in other manners. For example, the device 400 may be adapted to be secured to the hip stem 922 using cement, such as a bone cement. Such an embodiment is shown in FIG. 56, which is a perspective cross-sectional view of an embodiment of the device 500, illustrating cement ports 518 and cement receiving areas 520 of the device 500. The cement receiving areas 520 are designed to receive a volume of cement, such as bone cement. The cement ports 518 may extend through the body 502 from the outer surface 512 to the inner surface 510 within the aperture 504. The cement ports 518 may be in communication with the cement receiving areas 520, which are sized and shaped to receive a volume of cement. When the device 500 is positioned about the neck 924 of the hip stem 922, the cement receiving areas 520 are spaced apart from the neck 924, so that cement injected through the cement ports 518 can become positioning in the cement receiving areas 520 to secure the device 500 against the hip stem 922, such as about the stem flats 938 in the area of the neck 924. In other embodiments, the device may be molded to mate with the hip stem. For example, FIG. 57 is a cross-sectional view of another embodiment of a drug delivery device 600, illustrating the device implanted about a tapered hip stem 922. As shown, the device 600 may be molded such that an inner profile of the device 600 matches an outer profile of the tapered hip stem 922. In embodiments, the inner surface of the device 600 circumscribing the aperture may include a plastic material that is relatively soft in comparison to the rest of the body. The plastic material may form an inner liner that deforms, such as when the aperture receives the hip stem 922, to assist in securing the device 600 about the hip stem 922.

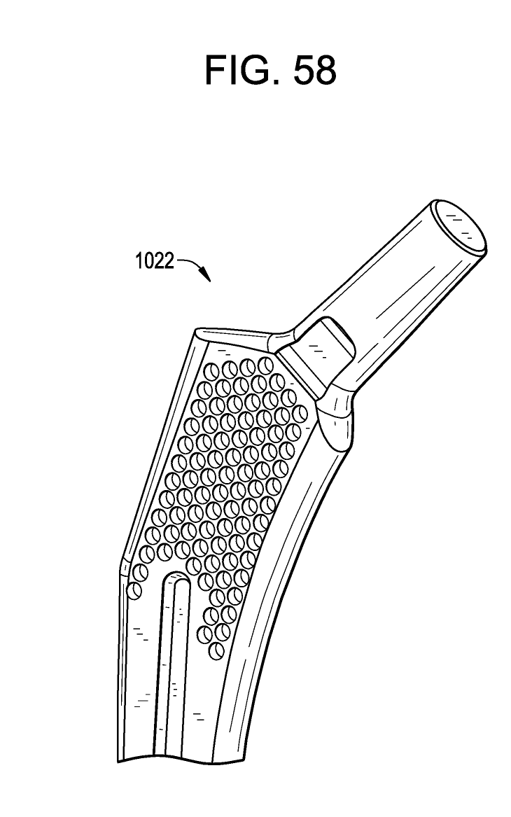

FIG. 58 is a cross-sectional view of an embodiment of a tapered hip stem 1022, which includes multiple discrete reservoirs for holding a drug formulation. The tapered hip stem 1022 can be used alone or in combination with any of the devices described above.

Various embodiments of the drug delivery device described above may be useful in drug therapy. In one case, the drug delivery devices may be used in at least part of an antibiotic treatment for a orthopedic surgery patient, in either prophylaxis or treatment of an infection. In one embodiment, the treatment provided by the drug delivery device may be over a relatively short term, such as a term of up to three months, in association with a hip replacement procedure, such as a cement-less total hip arthroplasty (THA) procedure or other THA procedures. Such short term prophylactic antibiotic therapy may reduce the incidence of post-operative infection associated with the implantation procedure.

Each of the devices described above may be formed of, for example, a biocompatible metal, a ceramic, a degradable or non-degradable polymer, or combinations thereof. In some embodiments, the body may be formed from a core covered in a drug-eluting coating. The coating may be a blend of a polymer and a drug, such that the drug is released from the polymer, for example by diffusion and/or surface dissolution/erosion when the polymer is bioerodible or biodegradable. In embodiments in which the body has a core, the reservoirs may be omitted. Alternatively, the body may have the reservoirs and the drug-eluting coating.

The device may be designed from permanent materials, such as metals or ceramics, or resorbable materials. In embodiments in which the device is formed from resorbable materials, the device may release the drug formulation of a period of time that is relatively shorter than the period of time over which the device resorbs. For example, the device may deliver all or substantially all of the drug formulation over a period of about three months, and the device may resorb over a period of about one year.

Making the Devices

The basic methods of fabricating and assembling the elongated body portion described herein are known or can be readily adapted from techniques known in the art. Reservoirs may be created in the device body simultaneously with formation of the device body, or they may be formed in the device body after the device body is made.

Representative fabrication techniques include conventional material forming processes, such as those known in the art for forming contoured metal, ceramic, or polymeric articles. In some embodiments, the devices may be made using methods that include MEMS fabrication processes, microfabrication processes, or other micromachining processes, various drilling techniques (e.g., laser, mechanical, and ultrasonic drilling), electrical discharge machining (EDM), and build-up or lamination techniques, such as LTCC (low temperature co-fired ceramics). Microfabrication methods include lithography and etching, injection molding and hot embossing, electroforming/electroplating, microdrilling (e.g., laser drilling), micromilling, electrical discharge machining (EDM), photopolymerization, surface micromachining, high-aspect ratio methods (e.g., LIGA), micro stereo lithography, silicon micromachining, rapid prototyping, and DEEMO (Dry Etching, Electroplating, Molding). Reservoirs may be fabricated into metal body portions by techniques known in the art, including laser etching, laser jet etching, micro-EDM, oxide film laser lithography, and computerized numerical control machining.

The surface of the reservoir optionally may be treated or coated to alter one or more properties of the surface. Examples of such properties include hydrophilicity/hydrophobicity, wetting properties (surface energies, contact angles, etc.), surface roughness, electrical charge, release characteristics, and the like.

U.S. Pat. No. 6,808,522; U.S. Pat. No. 6,123,861; U.S. Pat. No. 6,527,762; and U.S. Pat. No. 6,976,982, which are hereby incorporated by reference, describe micromolding and other techniques for making certain reservoir device bodies and caps. These methods may be modified and adapted, based on the teachings herein, to make the implant devices described herein.

Implanting/Using the Devices

The drug delivery device typically is implanted during the surgical procedure for implanting the prosthetic device. The surgical procedure may be a least invasive procedure that is performed, for example, through a 2 to 3 inch incision. The procedure typically involves preparing the bone tissue, which may include removing or resurfacing part of the bone and other tissues. The prosthetic device is then implanted in the surgically prepared bone in a conventional manner.

In one embodiment, the drug delivery device may be secured to the prosthetic device before the prosthetic device is implanted at the tissue site. In another embodiment, the drug delivery device may be secured to the prosthetic device after the prosthetic device is implanted at the tissue site. The device may be implanted into a patient (such as a human or other vertebrate animal) using standard surgical implantation techniques.

Robotic guidance systems or imaging techniques may be employed to confirm proper positioning of the implant.

Following implantation into the patient, the devices releases one or more drugs locally at the tissue site of implantation at a preselected delivery profile (e.g., dosing schedule) based on the design of the particular device as prescribed by the patient's physician. In one embodiment, the devices store and release an effective amount of at least one drug formulation over an extended period, e.g., between about 1 month and about 3 months.

Release of the drug(s) may be passively controlled as described hereinabove.

Modifications and variations of the methods and devices described herein will be obvious to those skilled in the art from the foregoing detailed description. Such modifications and variations are intended to come within the scope of the appended claims.

We claim:

1. An implantable drug delivery device adapted to be installed at or adjacent an implantable orthopedic prosthetic device inserted onto a surgically prepared bone section of a human or other animal, comprising:
a body having a core formed from a non-biodegradable material formed in the shape of a sleeve and having an inner surface and an outwardly directed outer surface; and
wherein said outer surface has a drug formulation for release thereof into the body of the animal following implantation of the prosthetic device within the body of the animal.

2. The drug delivery device of claim 1, wherein the body defines an open shape having free ends.

3. The drug delivery device of claim 1, wherein the body defines a closed annular shape.

4. The drug delivery device of claim 1, wherein the outer surface defines at least one pocket for holding the drug formulation.

5. The drug delivery device of claim 1, wherein the body core is formed of a material belonging to the group of materials consisting of: metal, metal alloy, ceramic, and polymers.

6. The drug delivery device of claim 1, wherein said drug formulation comprises an antibiotic.

7. The drug delivery device of claim 1, wherein the body further comprises a fixation means chosen from the group of fixation means consisting of a hole adapted to receive a screw, a pin, a suture, a cement, or a glue; a detent adapted to engage a groove; an indentation adapted to receive a cement or glue; a snap fitting; a pressure fitting; a screw fitting; a screw; a pin; a suture; a cement; a glue; and a groove.

8. The drug delivery device of claim 1, wherein said prosthetic device is a portion of a hip or knee replacement joint.

9. The drug delivery device of claim 1, wherein said prosthetic device is a hip replacement joint having a femoral portion including a hip stem having a neck;
wherein a portion of the body is adapted to be cemented to at least a portion of one or both of the following: said hip stem and said surgically prepared bone section; and
wherein said body in the shape of a sleeve defines an aperture adapted to receive a portion of said hip stem in confronting relation around three sides of said hip stem.

10. The drug delivery device of claim 1, wherein said prosthetic device is a hip replacement joint having a femoral portion including a hip stem having a neck;
wherein the body is adapted to receive said hip stem with said inner surface of the body in surrounding relation to said neck.

11. The drug delivery device of claim 2, wherein said prosthetic device is a knee replacement joint having an articular surface;
wherein said body is adapted to engage a peripheral portion of said articular surface; and
wherein said sleeve has an approximate C-shaped extent, portions of said sleeve being smooth in regions to be overlaid by muscle or tendon tissue.

12. An implantable drug delivery device adapted to be installed at or adjacent an implantable orthopedic prosthetic device inserted onto a surgically prepared bone section of a human or other animal, comprising:
a body having a radially inwardly directed inner surface defining an aperture, and a radially outwardly directed outer surface, and said body defining an open shape having two free ends;
said body having a thickness dimension defined in a generally radial direction extending from said inner surface to said outer surface;
wherein said aperture extends over the length of the thickness dimension; and wherein said outer surface holds a drug formulation for release thereof into the body of the animal following implantation of the prosthetic device within the body of the animal.

13. The drug delivery device of claim 12, wherein the outer surface defines at least one pocket for holding said drug formulation.

14. The drug delivery device of claim 12, wherein the body is formed of a material belonging to the group of materials consisting of: metal, metal alloy, ceramic, and polymers.

15. The drug delivery device of claim 12, wherein the at least one pocket contains the drug formulation, said drug formulation comprising an antibiotic.

16. The drug delivery device of claim 12, wherein the body further comprises a fixation means chosen from the group of fixation means consisting of a hole adapted to receive a screw, a pin, a suture, a cement, or a glue; a detent adapted to engage a groove; an indentation adapted to receive a cement or glue; a snap fitting; a pressure fitting; a screw fitting; a screw; a pin; a suture; a cement; a glue; and a groove.

17. The drug delivery device of claim 12, wherein said prosthetic device is a portion of a hip or knee replacement joint.

18. The drug delivery device of claim 12, wherein said prosthetic device is a hip replacement joint having a femoral portion including a hip stem having a neck;
wherein said body is adapted to be cemented to at least a portion of said hip stem or said surgically prepared bone section; and
wherein said aperture is adapted to receive a portion of said hip stem in confronting relation around three sides of said hip stem.

19. An implantable drug delivery device adapted to be installed on an implantable orthopedic prosthetic device inserted onto a surgically prepared bone section of a human or other animal, comprising:
a body having a first wall and an outer surface defining at least one pocket for holding a drug formulation, said first wall adapted to be cemented to at least one of a portion of said implantable prosthetic device or said surgically prepared bone section;
an aperture formed in said body and extending through said body, said aperture adapted to receive a portion of said implantable prosthetic device; and
said at least one pocket holds a drug formulation for release thereof into the body of the animal following implantation of the prosthetic device within the body of the animal.

20. The drug delivery device of claim 19, wherein said drug formulation comprises an antibiotic.

21. The drug delivery device of claim 19, wherein the body further comprises a fixation means chosen from the group of fixation means consisting of a hole adapted to receive a screw, a pin, a suture, a cement, or a glue; a detent adapted to engage a groove; an indentation adapted to receive a cement or glue; a snap fitting; a pressure fitting; a screw fitting; a screw; a pin; a suture; a cement; a glue; and a groove.

22. The drug delivery device of claim 19, wherein said prosthetic device is a portion of a hip or knee replacement joint.

23. The drug delivery device of claim 19, wherein said prosthetic device is a hip replacement joint having a femoral portion including a hip stem having a neck;
wherein said first wall is adapted to be cemented to at least a portion of said hip stem or said surgically prepared bone section; and
wherein said aperture is adapted to receive a portion of said hip stem in confronting relation around three sides of said hip stem.

24. A method of delivering a drug to a human or other animal having an implantable orthopedic prosthetic device, comprising:
implanting the orthopedic prosthetic device onto a surgically prepared bone section of the human or other animal;
providing an implantable drug delivery device having an aperture; and
surrounding a portion of the implanted prosthetic device with the aperture of the implantable drug delivery device in a manner preventing relative motion between the prosthetic device and the drug delivery device.
